(19) United States
(12) Patent Application Publication
(10) Pub. No.: US 2024/0308982 A1
(43) Pub. Date: Sep. 19, 2024

(54) DEGRADERS OF WILD-TYPE AND MUTANT FORMS OF LRRK2 AND USES THEREOF

(71) Applicant: DANA-FARBER CANCER INSTITUTE, INC., Boston, MA (US)

(72) Inventors: Nathanael S. Gray, Stanford, CA (US); John Hatcher, Boston, MA (US)

(73) Assignee: DANA-FARBER CANCER INSTITUTE, INC., Boston, MA (US)

(21) Appl. No.: 18/576,068

(22) PCT Filed: Jul. 7, 2022
(86) PCT No.: PCT/US2022/073517

§ 371 Date: Jan. 2, 2024

Related U.S. Application Data
(60) Provisional application No. 63/219,629, filed on Jul. 8, 2021.

(51) Int. Cl. C07D 401/14; A61K 31/506; C07D 417/14

(52) U.S. Cl. CPC C07D 401/14; A61K 31/506; C07D 417/14

(57) ABSTRACT

Disclosed are bifunctional compounds and pharmaceutically acceptable salts and stereoisomers thereof that that are potent and selective degraders of LRRK2. Also disclosed are pharmaceutical compositions containing same, and methods of making and using the compounds to treat diseases and disorders associated with LRRK2. In some embodiments, the disease or disorder is a neurodegenerative disorder. Parkinson's disease (PD), an inflammatory bowel disease (IBD), Crohn's disease (CD), leprosy (Hansen's disease), tuberculosis, meningioma, breast cancer, lung cancer or thyroid cancer.

### RELATED APPLICATIONS

This application claims the benefit of priority under 35 U.S.C. § 119(e) to U.S. Provisional Application No: 63/219,629, filed Jul. 8, 2021, which is incorporated herein by reference in its entirety.

### SEQUENCE LISTING

The instant application contains a Sequence Listing which has been submitted electronically in XML format and is hereby incorporated by reference in its entirety. Said XML copy, created on Jun. 30, 2022, is named 52095-728001WO_ST26.xml and is 4.34 KB bytes in size.

### BACKGROUND OF THE DISCLOSURE

Parkinson's disease (PD) is a movement disorder resulting from progressive loss of dopamine producing neurons. It is the second most common neurodegenerative disease in the world and affects over 1 million Americans. More than 60,000 patients are newly diagnosed each year (Gandhi et al., J. Neurosci. Res. 87:1283-1295 (2009); Dorsey et al., Neurology 68:384-386 (2007)). Symptoms associated with Parkinson's disease include motor impairment, tremor, bradykinesia, instability, and other movement related disorders. There are also non-motor symptoms such as cognitive dysfunction, autonomic dysfunction, and sleep disruption. These symptoms greatly reduce the quality of life of those suffering from Parkinson's disease.

Recent genetic studies have revealed an underlying genetic cause in at least 10% of all PD cases, which provide new opportunities for the discovery of molecularly targeted therapeutics that may ameliorate neurodegeneration (Daniëls et al., Neurosignals 19:1-15 (2011)). Insofar as the genes associated with PD are concerned, leucine-rich repeat kinase 2 (LRRK₂) having a missense mutation, G2019S, is frequently found in both familial and sporadic PD cases. (Healy et al., Lancet Neurol. 7:583-590 (2008), Dächsel et al., Neurol. 67:542-547 (2010), Lee et al., Trends Pharmacol. Sci. 33(7):365-373 (2012), Liu et al., Hum. Mol. Genet. 20:3933-3942 (2011)). The G2019S mutation increases kinase activity, which may result in activation of the neuronal death signal pathway (Greggio et al., ASN Neuro 1(1):e00002 (2009), Kumar et al., Expert Rev. Mol. Med. 13:e20 (2011)). Transgenic G2019S LRRK₂ mice aged to 12-16 months displayed progressive degeneration of the substantia nigra pars compacta (SNpc) dopaminergic neurons and Parkinson's phenotypes of motor dysfunction, suggesting that this mutation may be functionally relevant to the disease (Chen et al., Cell Death Differ. 19(10): 1623-33 (2012)).

The state of LRRK₂ inhibitors has been recently reviewed, providing a summary of potency versus wild type and G2019S and A2016T mutations, brain permeability, and pharmacokinetic data, however, the reported compounds have not yet advanced beyond preclinical testing. (Atashrazm and Dzamko Clin. Pharm.: Advances and Applications 8 177-189 (2016)). Another recent review similarly provides an overview of data associated with LRRK₂ inhibitors, however, also noted are recent studies that point to potential undesired effects in peripheral tissue such as lung and kidney. (Taymans and Greggio, Current Neuropharm. 14, 214-225 (2016)).

Selective LRRK₂ degraders offer potential of providing advantages over existing LRRK₂ inhibitors, including: 1) effective intracellular concentrations of degraders are significantly lower than for conventional kinase antagonists because data suggests that these degraders act in a catalytic fashion (i.e. a single degrader molecule can induce degradation of multiple target proteins); 2) pharmacodynamic effects of the degraders are dictated by protein resynthesis rates similar to what is observed for covalent inhibitors, because degraders lead to complete elimination of the protein by the proteasome; 3) kinase degradation addresses tyrosine kinase inhibitor (TKI) resistance imparted by intrinsic ‘scaffolding’ functions of kinases; and 4) de novo resistance mutations to selective degraders of LRRK₂ are less likely to emerge, given that efficient degradation can be achieved even with lower affinity warheads (Churcher, I., J. Med. Chem. 61 (2), 444-452 (2018)).

### SUMMARY OF THE DISCLOSURE

A first aspect of the present disclosure is directed to a bifunctional compound having a structure represented by formula (Ia) or formula (Ib):

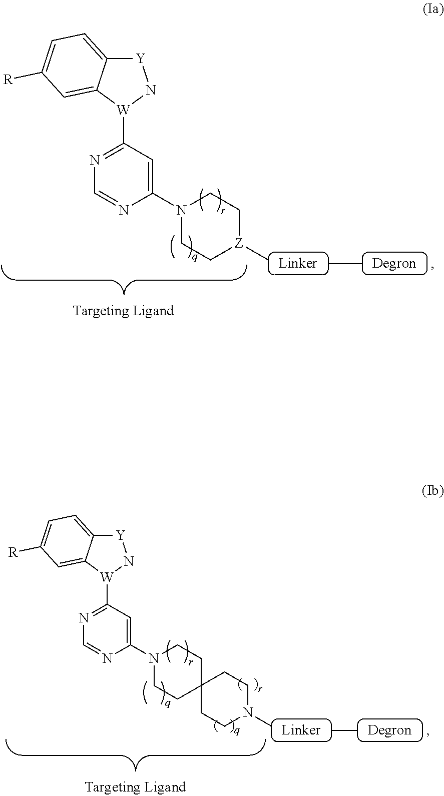

or a pharmaceutically acceptable salt or stereoisomer thereof, wherein —W—N—Y— is —C═N—NH— or —N—N═CH—; R is MeO—, EtO—, i-PrO—,

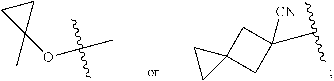

Z is N or CH; each occurrence of q and r is independently 0 or 1; the targeting ligand binds leucine-rich repeat kinase 2 (LRRK₂); the degron (“Degron”) represents a moiety that binds an E3 ubiquitin ligase; and the linker (“Linker”) provides a covalent attachment between the targeting ligand and the degron.

Another aspect of the present disclosure is directed to a pharmaceutical composition that includes a therapeutically effective amount of a compound of formula (Ia) or formula (Ib) or a pharmaceutically acceptable salt or stereoisomer thereof, and a pharmaceutically acceptable carrier.

A further aspect of the present disclosure is directed to methods of treating a disease or disorder that is characterized or mediated by aberrant activity of LRRK₂ or a mutant form thereof, by administering to a subject in need thereof a therapeutically effective amount of the bifunctional compound of formula (Ia) or formula (Ib) or pharmaceutically acceptable salt or stereoisomer thereof. In some embodiments, the disease or disorder is characterized or mediated by aberrant activity of a wild-type LRRK₂. In some embodiments, the disease or disorder is characterized or mediated by aberrant activity of a LRRK₂ mutant such as the G2019S mutant.

In some embodiments, the disease or disorder is a neurodegenerative disorder. In some embodiments, the disease or disorder is Parkinson's disease (PD). In some embodiments, the disease or disorder is an inflammatory bowel disease (IBD). In some embodiments, the disease or disorder is Crohn's disease (CD). In some embodiments, the disease or disorder is leprosy (Hansen's disease). In some embodiments, the disease or disorder is tuberculosis. In some embodiments, the disease or disorder is a meningioma. In some embodiments, the disease or disorder is cancer. In some embodiments, the cancer is breast cancer, lung cancer or thyroid cancer.

A further aspect of the present disclosure is directed to methods of reducing the levels of LRRK₂ in a cell, either in vitro or in vivo, comprising contacting the cell with an effective amount of the bifunctional compound of formula (Ia) or formula (Ib) or pharmaceutically acceptable salt or stereoisomer thereof.

As demonstrated in the working examples, compounds of the present disclosure are potent and selective degraders of LRRK2.

### BRIEF DESCRIPTION OF THE DRAWINGS

FIG. 1 is a set of immunoblots of 448T cells treated with the indicated compounds (1), (2), and (3) at concentrations ranging from 0 to 1000 nM.

### DETAILED DESCRIPTION OF THE DISCLOSURE

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

With respect to compounds of the present disclosure, and to the extent the following terms are used herein to further describe them, the following definitions apply.

As used herein, the term “alkyl” refers to a saturated linear or branched-chain monovalent hydrocarbon radical. To the extent not defined otherwise for any particular group in the compounds of formula (Ia) and formula (Ib), in one embodiment, the alkyl radical is a C₁-C₁₈ group. In other embodiments, the alkyl radical is a C₁-C₁₂, C₁-C₈, C₁-C₆, C₁-C₅, C₁-C₄ or C₁-C₃ group. Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. To the extent not defined otherwise for any particular group in the compounds of formula (Ia) and formula (Ib), the alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. To the extent not defined otherwise for any particular group in the compounds of formula (Ia) and formula (Ib), an alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “alkoxylene” refers to a saturated monovalent aliphatic radicals of the general formula (—O—CₙH₂ₙ—) where n represents an integer (e.g., 1, 2, 3, 4, 5, 6, or 7) and is inclusive of both straight-chain and branched-chain radicals. To the extent not defined otherwise for any particular group in the compounds of formula (Ia) and formula (Ib), the alkoxylene chain may be attached to the rest of the molecule through a single bond and to the radical group through a single bond. In some embodiments, the alkoxylene group contains one to 3 carbon atoms (—O—C₁-C₃ alkoxylene). In other embodiments, an alkoxylene group contains one to 5 carbon atoms (—O—C₁-C₅ alkoxylene).

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. To the extent not defined otherwise for any particular group in the compounds of formula (Ia) and formula (Ib), cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). To the extent not defined otherwise for any particular group in the compounds of formula (Ia) and formula (Ib), the term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. To the extent not defined otherwise for any particular group in the compounds of formula (Ia) and formula (Ib), in some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring. The structure of any aryl group that is capable of having double bonds positioned differently is considered so as to embrace any and all such resonance structures.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). To the extent not defined otherwise for any particular group in the compounds of formula (Ia) and formula (Ib), the term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3-to 7-membered monocycles having one or more heteroatoms selected from nitrogen, sulfur or oxygen. In some embodiments, heterocyclyl includes 4-to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1 ]octanyl, 2-azabicyclo[2.2.2]octanyl, 8-azabicyclo[2.2.2]octanyl, 7-oxabicyclo[2.2.1 ]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring. The structure of any heteroaryl group that is capable of having double bonds positioned differently is considered so as to embrace any and all such resonance structures.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s) in the compounds of formula (Ia) and formula (Ib), representative examples of substituents may include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the targeting ligand and the targeted protein, which in this disclosure is LRRK2, typically refers to an inter-molecular interaction that is preferential (also referred to herein as “selective”) in that binding of the targeting ligand with other proteins present in the cell, including other LRRK isoforms, is substantially less and functionally insignificant, at least from the standpoint of degradation. The terms “selective” and “selectivity” refer to the ability of the bifunctional compound to discriminate between molecular targets. A selective LRRK2 degrader described herein may have a DC₅₀ (half maximal degradation concentration) for LRRK2 activity that is at least about 1, 2, 3, 4, 5, 6, 7, 8, 9, or 10-fold lower than the DC₅₀ for one or more of other LRRK family members (e.g., LRRK1), and other kinases. Thus, even though various bifunctional compounds of the present disclosure bind to other LRRK proteins, albeit with similar or much less affinity, they show selective degradation of LRRK2.

The term “binding” as it relates to interaction between the degron and the E3 ubiquitin ligase, typically refers to an inter-molecular interaction that may or may not exhibit an affinity level that equals or exceeds that affinity between the targeting ligand and LRRK2, but nonetheless wherein the affinity is sufficient to achieve recruitment of the ligase to the targeted degradation and the selective degradation of LRRK2.

An aspect of the present disclosure is directed to a bifunctional compound having a structure represented by formula (Ia) or formula (Ib):

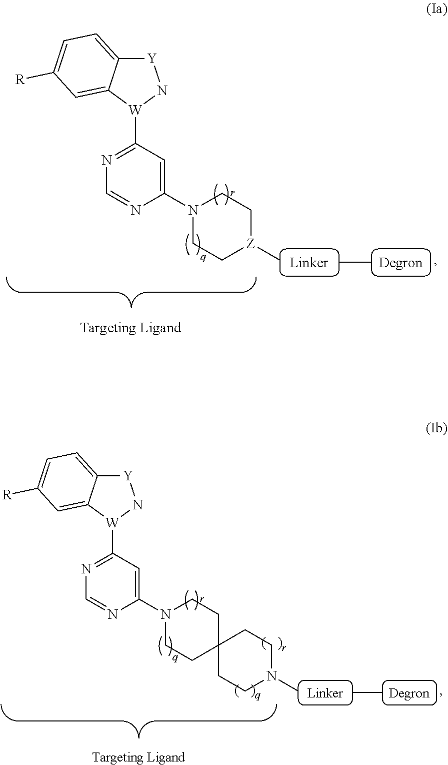

or a pharmaceutically acceptable salt or stereoisomer thereof, wherein —W—N—Y— is —C═N—NH— or —N—N═CH—; R is MeO—, EtO—, i-PrO—,

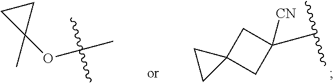

Z is N or CH; each occurrence of q and r is independently 0 or 1; the targeting ligand binds leucine-rich repeat kinase 2 (LRRK2), the degron (“Degron”) represents a moiety that binds an E3 ubiquitin ligase, and the linker (“Linker”) provides a covalent attachment between the targeting ligand and the degron. In some embodiments, q and r=0. In some embodiments, q and r=1.

In some embodiments, the degron binds a Von Hippel-Lindau (VHL) tumor suppressor. In some embodiments, the degron is represented by any one of the following structures:

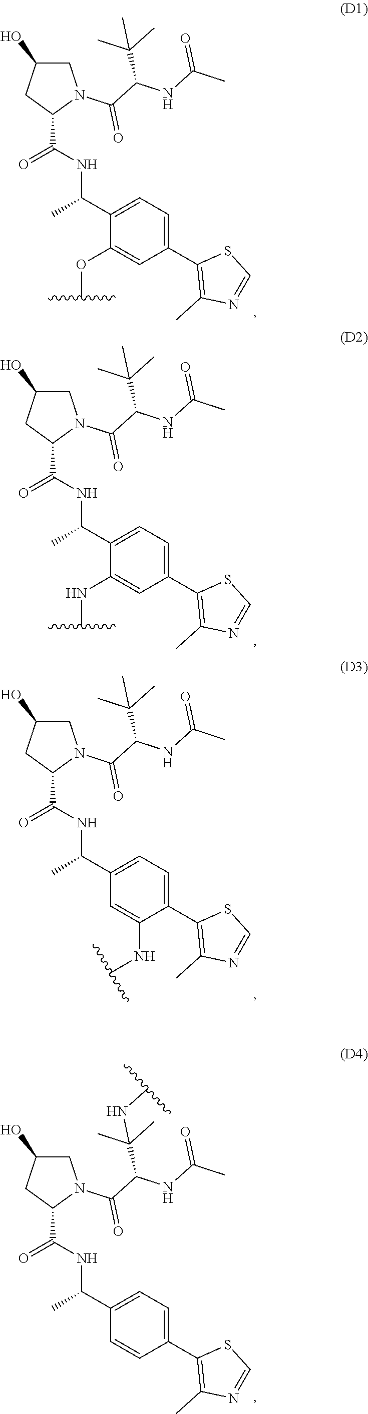

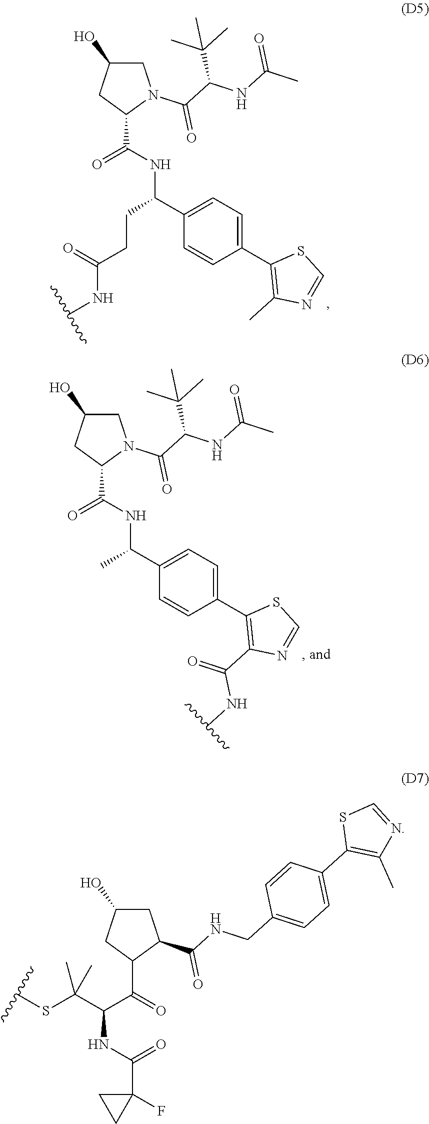 [SMILES: *NC(=O)CC[C@H](NC(=O)[C@@H]1C[C@@H](O)CN1C(=O)[C@@H](NC(C)=O)C(C)(C)C)c1ccc(-c2scnc2C)cc1 ; *NC(=O)c1ncsc1-c1ccc([C@H](C)NC(=O)[C@@H]2C[C@@H](O)CN2C(=O)[C@@H](NC(C)=O)C(C)(C)C)cc1 ; *SC(C)(C)[C@H](NC(=O)C1(F)CC1)C(=O)C1C[C@H](O)C[C@H]1C(=O)NCc1ccc(-c2scnc2C)cc1]

Yet other degrons that bind VHL and which may be suitable for use as degrons in the present disclosure are disclosed in U.S. Patent Application Publication 2017/0121321 A1.

In some embodiments, the degron binds an inhibitor of apoptosis protein (IAP). In some embodiments, the degron is represented by any one of the following structures:

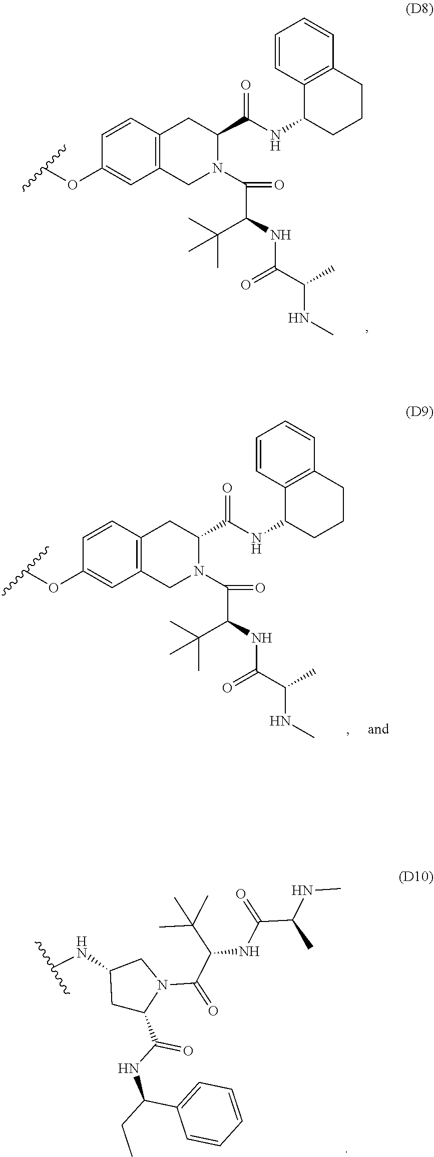

Yet other degrons that bind IAPs and which may be suitable for use as degrons in the present disclosure are disclosed in International Patent Application Publications WO 2008/128171, WO 2008/016893, WO 2014/060768, WO 2014/060767, and WO 2015/092420. IAPs are known in the art to function as ubiquitin-E3 ligases.

In some embodiments, the degron binds cereblon (CRBN). In some embodiments, the degron is represented by any one of the following structures:

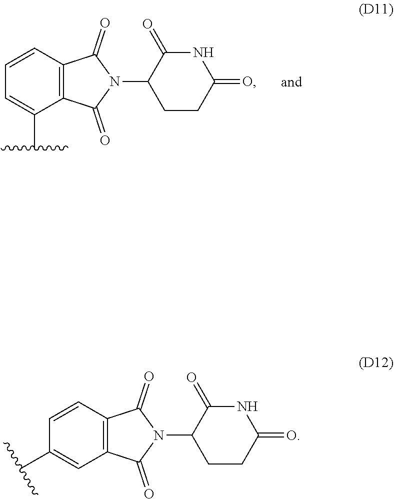 [SMILES: *c1cccc2c1C(=O)N(C1CCC(=O)NC1=O)C2=O ; "and" ; *c1ccc2c(c1)C(=O)N(C1CCC(=O)NC1=O)C2=O]

Yet other degrons that bind cereblon and which may be suitable for use as degrons in the present disclosure are described in U.S. Patent Application Publication 2018/0015087 (e.g., the indolinones such as isoindolinones and isoindoline-1,3-diones embraced by formulas IA and IA′ therein, and the bridged cycloalkyl compounds embraced by formulae IB and IB′ therein).

In some embodiments, the linker provides a covalent attachment between the targeting ligand and the degron. The structure of the linker may not be critical, provided it does not substantially interfere with the activity of the targeting ligand or the degron.

In some embodiments, the linker is represented by the structure:

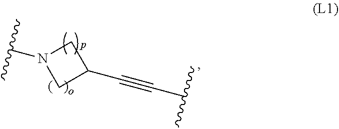

wherein o and p are independently 1, 2, or 3.

In some embodiments, the linker comprises an alkylene chain which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′), —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O), —S(O)N(R′)—, —N(R′)S(O)₂N(R′)—, —N(R′)S(O)N(R′)—, C₃-C₁₂ carbocyclene, 3-to 12-membered heterocyclene, 5-to 12-membered heteroarylene or any combination thereof, wherein R′ is H or C₁-C₆ alkyl; and the interrupting and the one or both terminating groups may be the same or different. In some embodiments, the alkylene chain comprises 1-6 alkylene units.

In some embodiments, the linker comprises a C₁ to C₃ alkylene chain which may be interrupted by, and/or terminate (at either or both termini) in at least one of —O—, —N(R′)—, —C≡C—, —C(O), 4-to 8-membered heterocyclene, C₃-C₈ cycloalkyl, or any combination thereof, wherein R′ is H or C₁-C₆ alkyl; and the interrupting and the one or both terminating groups may be the same or different. In some embodiments, the C₁ to C₃ alkylene chain terminates (at either or both termini) in at least one of —O—, —N(R′)—, —C≡C—, —C(O)—, or any combination thereof, wherein R′ is H or C₁-C₆ alkyl. In some embodiments, the linker is represented by any one of the structures:

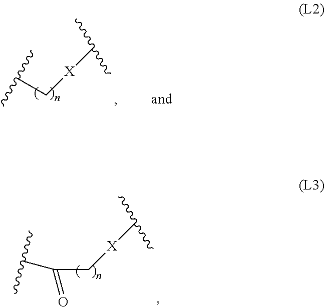

wherein n is 1, 2, or 3; and X is —NH—, —O— or —C≡C—.

In some embodiments, the C₁ to C₃ alkylene chain terminates (at either or both termini) in at least one of —C(O)—, 4-to 8-membered heterocyclene, or any combination thereof. In some embodiments, the linker is represented by the structure:

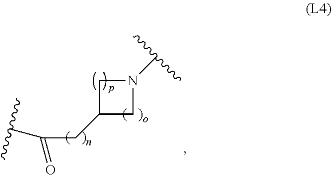

wherein n, o, and p are independently 1, 2, or 3.

In some embodiments, the C₁ to C₃ alkylene chain terminates (at either or both termini) in at least one of —C≡C—, 4-to 8-membered heterocyclene, or any combination thereof. In some embodiments, the linker is represented by the structure:

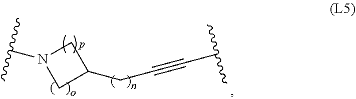

wherein n, o, and p are independently 1, 2, or 3.

In some embodiments, the C₁ to C₃ alkylene chain terminates (at either or both termini) in at least one 4-to 8-membered heterocyclene. In some embodiments, the linker is represented by any one of the structures:

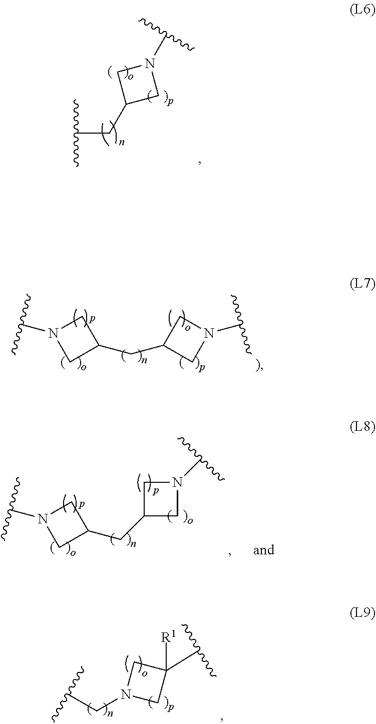

wherein each occurrence of n, o, and p is independently 1, 2, or 3; and R¹ is —H, —OH, —NH₂, —SH, or —SeH.

In some embodiments, the C₁ to C₃ alkylene chain is interrupted by a C₃-C₈ cycloalkyl.

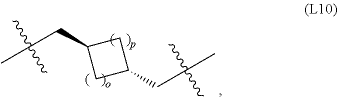

In some embodiments, the linker is represented by the structure: wherein o and p are independently 1, 2 or 3.

In some embodiments, the linker comprises a polyethylene glycol (PEG) chain which may terminate (at either or both termini) in at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, ——N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′), —N(R′)S(O)₂N(R′)—, —N(R′)S(O)N(R′)—, C₃₋₁₂ carbocyclene, 3-to 12-membered heterocyclene, 5- to 12-membered heteroarylene or any combination thereof, wherein R′ is H or C₁-C₆ alkyl; and the one or both terminating groups may be the same or different. In some embodiments, the polyethylene glycol chain comprises 1-6 PEG units.

In some embodiments, the bifunctional compound, or a pharmaceutically acceptable salt or stereoisomer thereof, is represented by any one of the structures:

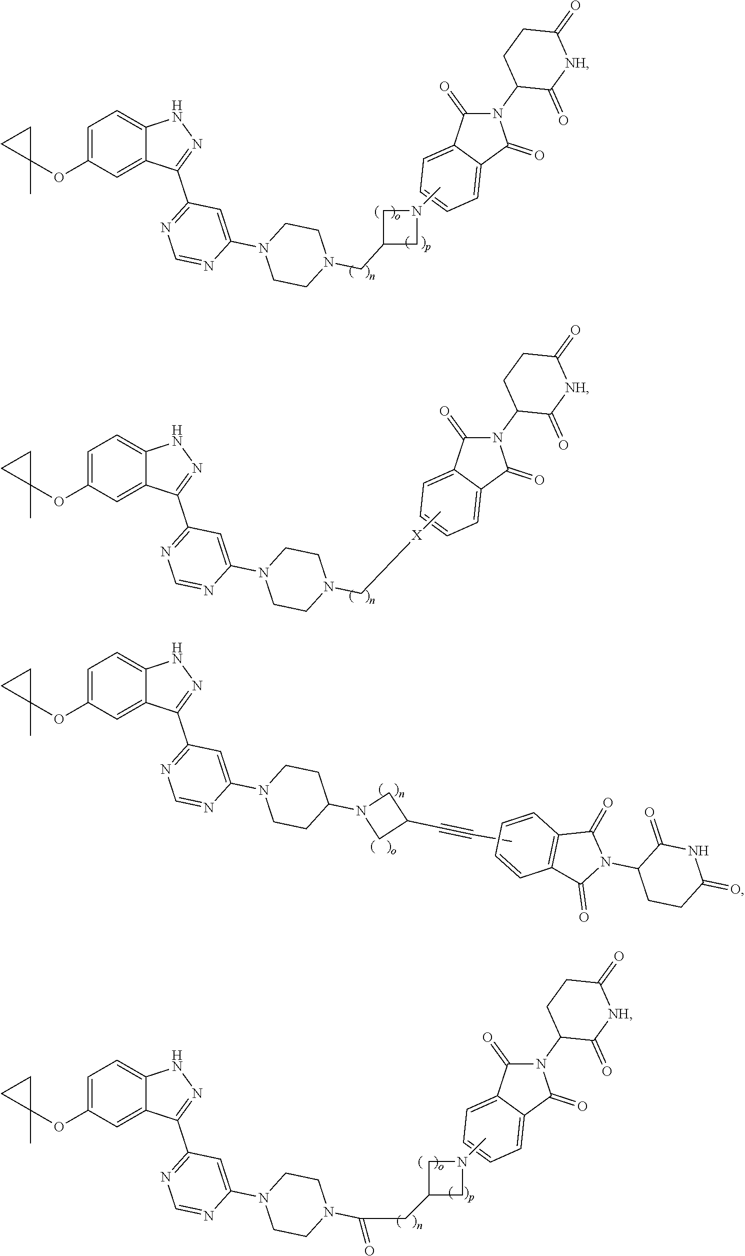

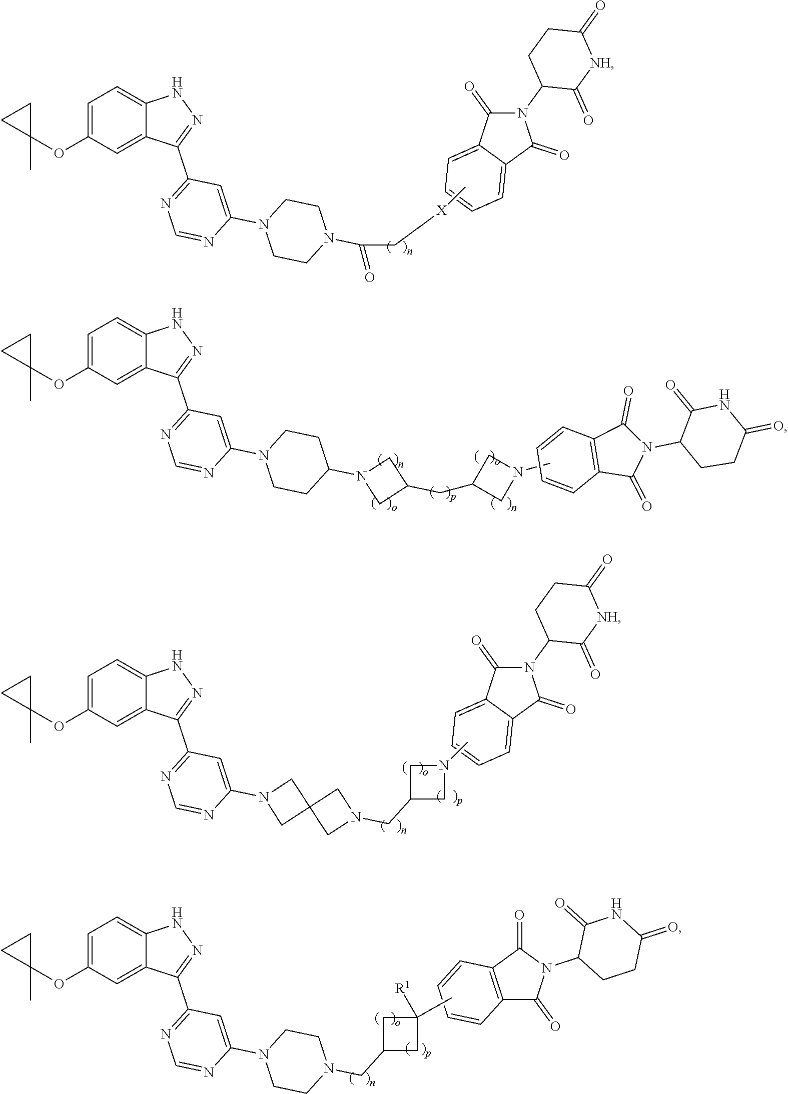

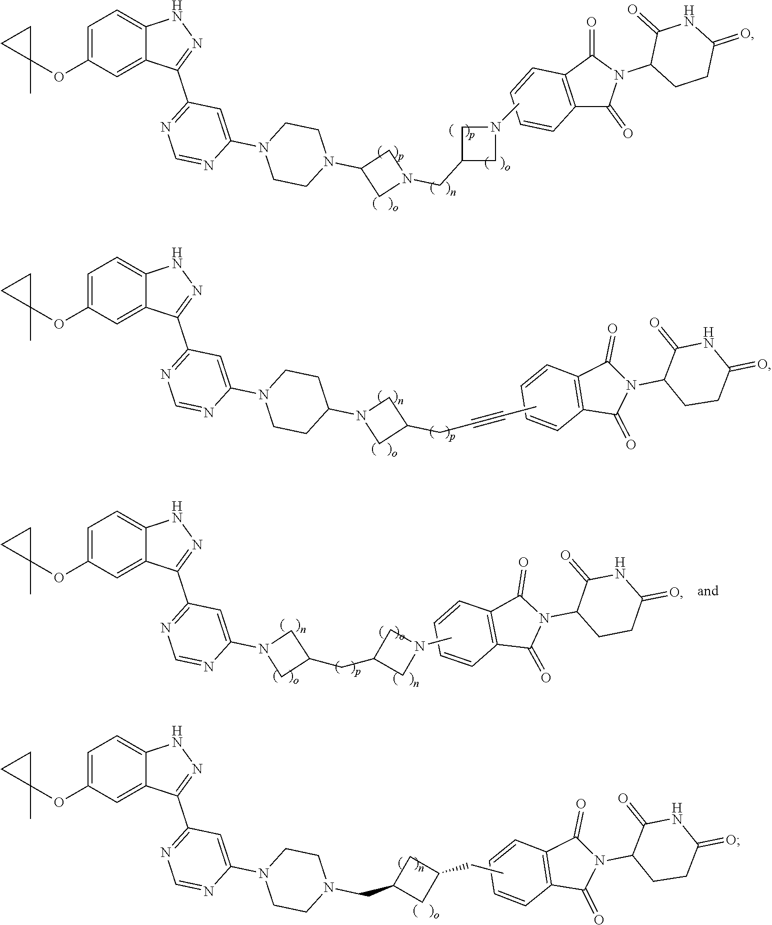

wherein each occurrence of n, o, and p is each independently 1, 2, or 3; X is —NH—, —O— or alkynyl; and R¹ is —H, —OH, —NH₂, —SH, or —SeH.

In some embodiments, the bifunctional compound or a pharmaceutically acceptable salt sterenisomer thereof, is renresented by any one of the structures: or

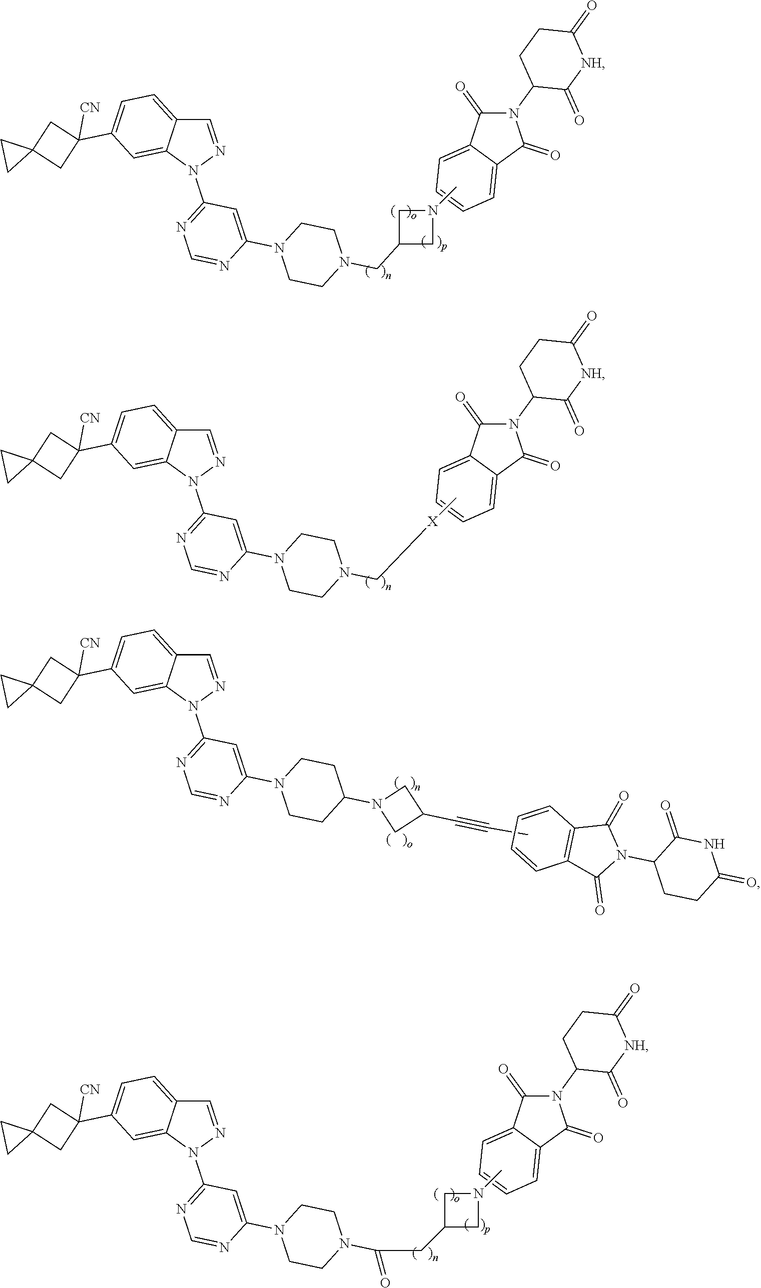

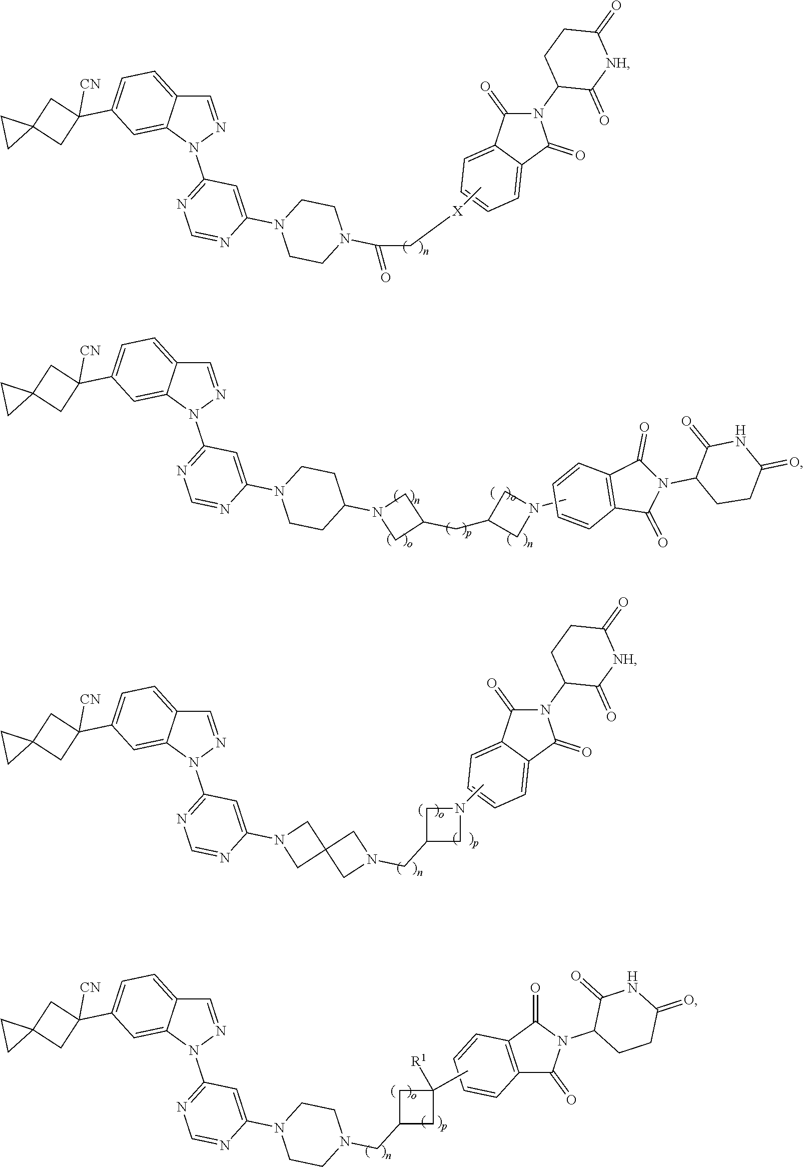

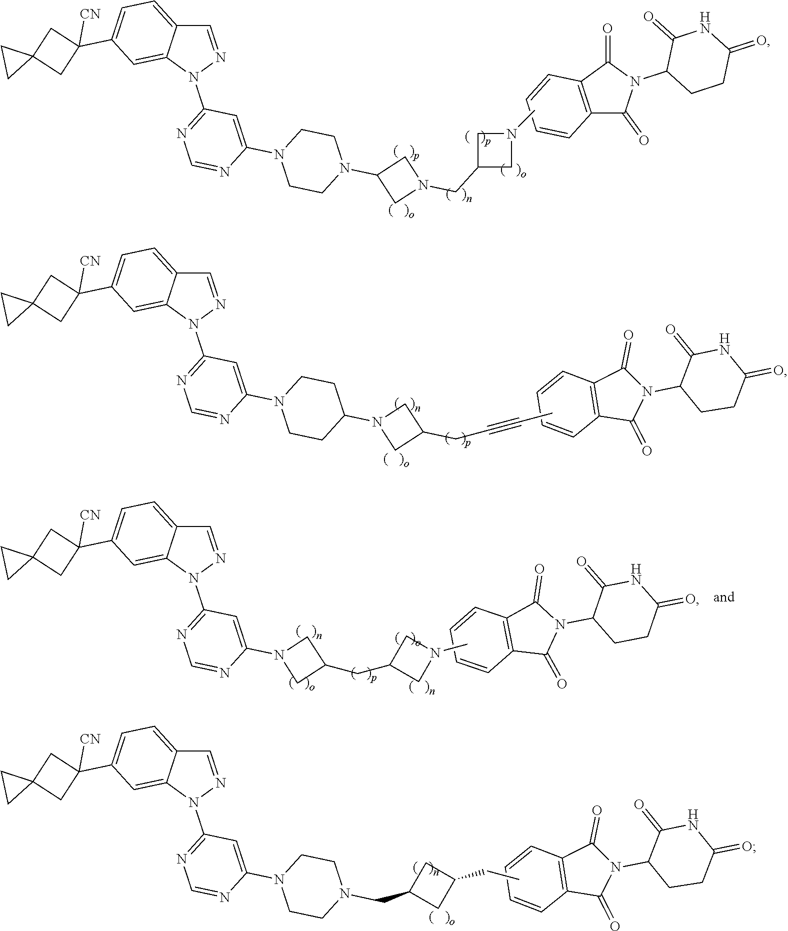

wherein n, o, and p are each independently 1, 2, or 3;
X is —NH—, —O— or alkynyl; and
R¹ is —H, —OH, —NH₂, —SH, or —SeH.

In some embodiments, the linker is represented by any one of the structures:

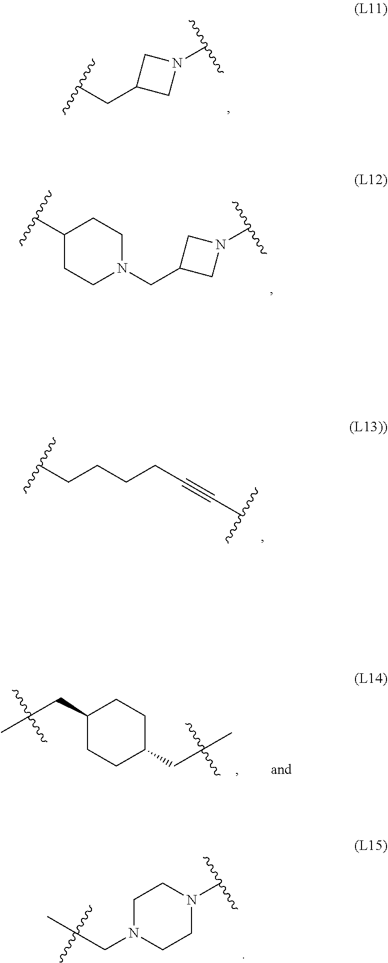 [SMILES: *CC1CN(*)C1 ; *C1CCN(CC2CN(*)C2)CC1 ; *C#CCCCC* ; *C[C@H]1CC[C@H](C*)CC1 ; *CN1CCN(*)CC1]

In some embodiments, the bifunctional compound, or a pharmaceutically acceptable salt or stereoisomer thereof, contains any one of the targeting ligands shown in formulas (Ia) and formula (Ib), any one of linkers L1-L15 and any one of degrons D1-D12.

In some embodiments, the bifunctional compound of formula (Ia) or formula (Ib), or a pharmaceutically acceptable salt or stereoisomer thereof, is:

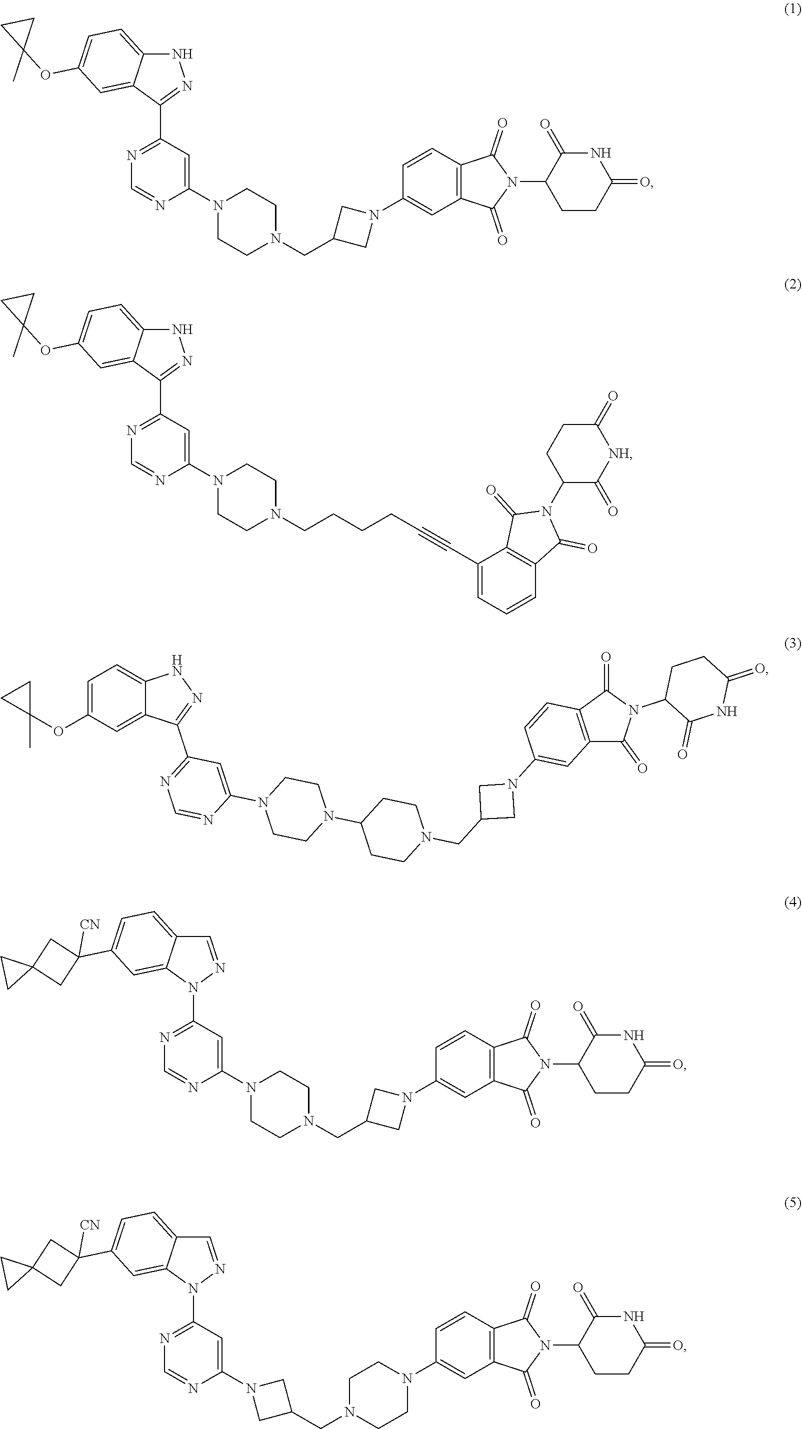 [SMILES: CC1(Oc2ccc3[nH]nc(-c4cc(N5CCN(CC6CN(c7ccc8c(c7)C(=O)N(C7CCC(=O)NC7=O)C8=O)C6)CC5)ncn4)c3c2)CC1 ; CC1(Oc2ccc3[nH]nc(-c4cc(N5CCN(CCCCC#Cc6cccc7c6C(=O)N(C6CCC(=O)NC6=O)C7=O)CC5)ncn4)c3c2)CC1 ; CC1(Oc2ccc3[nH]nc(-c4cc(N5CCN(C6CCN(CC7CN(c8ccc9c(c8)C(=O)N(C8CCC(=O)NC8=O)C9=O)C7)CC6)CC5)ncn4)c3c2)CC1 ; N#CC1(c2ccc3cnn(-c4cc(N5CCN(CC6CN(c7ccc8c(c7)C(=O)N(C7CCC(=O)NC7=O)C8=O)C6)CC5)ncn4)c3c2)CC2(CC2)C1 ; N#CC1(c2ccc3cnn(-c4cc(N5CC(CN6CCN(c7ccc8c(c7)C(=O)N(C7CCC(=O)NC7=O)C8=O)CC6)C5)ncn4)c3c2)CC2(CC2)C1]

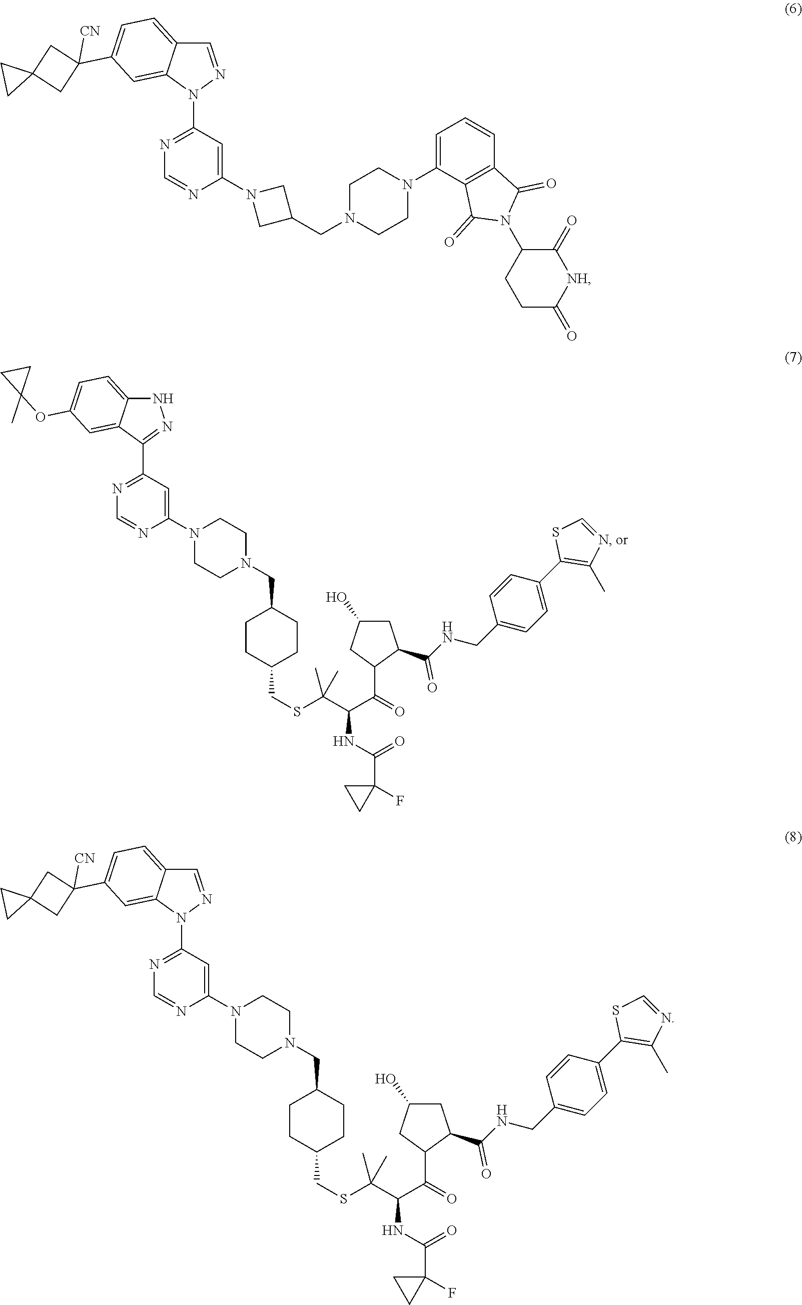

Bifunctional compounds of the present disclosure may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of a bifunctional compound of the present disclosure with a suitable acid or a base. Examples of pharmaceutically acceptable salts of the bifunctional compounds of this disclosure include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the disclosure can form pharmaceutically acceptable salts with various organic bases such as lysine, arginine, guanidine, diethanolamine or metformin. Suitable base salts include aluminum, calcium, lithium, magnesium, potassium, sodium, or zinc salts.

Bifunctional compounds of the present disclosure may have at least one chiral center and therefore may be in the form of a stereoisomer, which as used herein, embraces all isomers of individual compounds that differ only in the orientation of their atoms in space. The term stereoisomer includes mirror image isomers (enantiomers which include the (R—) or (S—) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R—) form is considered equivalent to administration of the compound in its (S—) form. Accordingly, the compounds of the present disclosure may be made and used in the form of individual isomers and substantially free of other isomers, or in the form of a mixture of various isomers, e.g., racemic mixtures of stereoisomers.

In some embodiments, the bifunctional compound is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e., ²H, may afford certain therapeutic advantages resulting from greater metabolic stability, for example, increased in vivo half-life or reduced dosage requirements, and thus may be advantageous in some circumstances.

The bifunctional compounds of the present disclosure may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other known techniques.

In some embodiments, the pharmaceutical composition comprises a co-crystal of a bifunctional compound. The term “co-crystal”, as used herein, refers to a stoichiometric multi-component system comprising a compound of the disclosure and a co-crystal former wherein the compound of the disclosure and the co-crystal former are connected by non-covalent interactions. The term “co-crystal former”, as used herein, refers to compounds which can form intermolecular interactions with a compound of the disclosure and co-crystallize with it. Representative examples of co-crystal formers include benzoic acid, succinic acid, fumaric acid, glutaric acid, trans-cinnamic acid, 2,5-dihydroxybenzoic acid, glycolic acid, trans-2-hexanoic acid, 2-hydroxycaproic acid, lactic acid, sorbic acid, tartaric acid, ferulic acid, suberic acid, picolinic acid, salicyclic acid, maleic acid, saccharin, 4,4′-bipyridine p-aminosalicyclic acid, nicotinamide, urea, isonicotinamide, methyl-4-hydroxybenzoate, adipic acid, terephthalic acid, resorcinol, pyrogallol, phloroglucinol, hydroxyquinol, isoniazid, theophylline, adenine, theobromine, phenacetin, phenazone, etofylline, and phenobarbital.

### Methods of Synthesis

In another aspect, the present disclosure is directed to methods for making bifunctional compounds of formulas Ia and Ib, and their pharmaceutically acceptable salts and stereoisomers. Broadly, the bifunctional compounds and their pharmaceutically acceptable salts and stereoisomers may be prepared by any process known to be applicable to the preparation of chemically related compounds. The bifunctional compounds of the present disclosure will be better understood in connection with the synthetic schemes that described in various working examples which illustrate non-limiting methods by which the compounds of the disclosure may be prepared.

### Pharmaceutical Compositions

Another aspect of the present disclosure is directed to a pharmaceutical composition that includes a therapeutically effective amount of a bifunctional compound or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present disclosure to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, bifunctional compounds of the disclosure and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999 Marchcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the bifunctional compounds are formulated for oral or intravenous administration (e.g., systemic intravenous injection).

Accordingly, bifunctional compounds of the disclosure may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the bifunctional compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bifunctional compounds of the disclosure may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bifunctional compounds of the disclosure may be administered in a local rather than systemic manner, for example, via injection of the conjugate directly into an organ, often in a depot preparation or sustained release formulation. In specific embodiments, long-acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges, and gels.

The bifunctional compounds of the disclosure may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bifunctional compounds of the disclosure may be formulated for topical administration which as used herein, refers to administration intradermally by disclosure of the formulation to the epidermis. These types of compositions are typically in the form of ointments, pastes, creams, lotions, gels, solutions and sprays.

Representative examples of carriers useful in formulating bifunctional compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a bifunctional compound or a pharmaceutically acceptable salt or stereoisomer thereof that is effective in producing the desired therapeutic response in a patient suffering from a disease or disorder that is characterized or mediated by aberrant LRRK2 activity. The term “therapeutically effective amount” thus includes the amount of a bifunctional compound or a pharmaceutically acceptable salt or stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or inhibits the growth of diseased cells, or reduces the amounts of LRRK2 in diseased cells.

The total daily dosage of the bifunctional compounds and usage thereof may be decided in accordance with standard medical practice, e.g., by the attending physician using sound medical judgment. The specific therapeutically effective dose for any particular subject will depend upon a variety of factors, including the following: the disease or disorder being treated and the severity thereof (e.g., its present status); the activity of the compound employed; the specific composition employed; the age, body weight, general health, sex and diet of the subject; the time of administration, route of administration, and rate of excretion of the compound employed; the duration of the treatment; drugs used in combination or coincidental with the specific compound employed; and like factors well known in the medical arts (see, for example, Hardman et al., eds., Goodman and Gilman's The Pharmacological Basis of Therapeutics, 10th Edition, McGraw-Hill Press, 155-173, 2001).

Bifunctional compounds of the disclosure may be effective over a wide dosage range. In some embodiments, the total daily dosage (e.g., for adult humans) may range from about 0.001 to about 1600 mg, from 0.01 to about 1000 mg, from 0.01 to about 500 mg, from about 0.01 to about 100 mg, from about 0.5 to about 100 mg, from 1 to about 100-400 mg per day, from about 1 to about 50 mg per day, from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, the compound may be administered at a dose in range from about 0.01 mg to about 200 mg/kg of body weight per day. In some embodiments, a dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day.

### Methods of Use

In some aspects, the present disclosure is directed to methods of treating diseases or disorders characterized by aberrant activity of LRRK2, comprising administering to a subject in need thereof a therapeutically effective amount of the bifunctional compound of formula (Ia) or formula (Ib), or a pharmaceutically acceptable salt or stereoisomer thereof, to a subject in need thereof.

In some aspects, the present disclosure is directed to methods of reducing the levels of LRRK2 in a cell, either in vitro or in vivo, comprising contacting the cell with an effective amount of the bifunctional compound of formula (Ia) or formula (Ib), or a pharmaceutically acceptable salt or stereoisomer thereof, to a subject in need thereof.

Broadly, the diseases or disorders that may be amenable to treatment with bifunctional compounds of the present disclosure involve aberrant (functionally abnormal) LRRK2 activity relative to a non-pathological state. A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health. In some embodiments, compounds of formula (Ia) or formula (Ib) may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

In some embodiments, the disease or disorder is characterized or mediated by aberrant activity of a wild-type LRRK2.

An exemplary wild-type LRRK2 polypeptide sequence is provided at NCBI Accession No. NP_940980, version NP_940980.4, incorporated herein by reference and set forth below (SEQ ID NO: 1):

```
(SEQ ID NO: 1)
      1 masgscqgce edeetlkkli vrlnnvqegk qietlvqile dllvftyser asklfqgkni
    61 hvpllivlds ymrvasvqqv gwsllcklie vcpgtmqslm gpqdvgndwe vlgvhqlilk
  121 mltvhnasvn lsviglktld llltsgkitl lildeesdif mlifdamhsf pandevqklg
  181 ckalhvlfer vseeqltefv enkdymills altnfkdeee ivlhvlhclh slaipcnnve
  241 vlmsgnvrcy nivveamkaf pmserigevs ccllhrltlg nffnilvlne vhefvvkavq
  301 qypenaalqi salsclallt etiflnqdle eknengendd egeedklfwl eacykaltwh
  361 rknkhvqeaa cwalnnllmy qnslhekigd edghfpahre vmlsmlmhss skevfqasan
  421 alstlleqnv nfrkillskg ihlnvlelmq khihspevae sgckmlnhlf egsntsldim
  481 aavvpkiltv mkrhetslpv qlealrailh fivpgmpees redtefhhkl nmvkkqcfkn
  541 dihklvlaal nrfignpgiq kcglkvissi vhfpdaleml slegamdsvl htlqmypddq
  601 eiqclglsli gylitkknvf igtghllaki lvsslyrfkd vaeiqtkgfq tilailklsa
  661 sfskllvhhs fdlvifhqms snimeqkdqq flnlcckcfa kvamddylkn vmleracdqn
  721 nsimveclll lgadangake gsslicqvce kesspklvel llnsgsreqd vrkaltisig
  781 kgdsqiisll lrrlaldvan nsiclggfci gkvepswlgp lfpdktsnlr kqtniastla
  841 rmviryqmks aveegtasgs dgnfsedvls kfdewtfipd ssmdsvfaqs ddldsegseg
  901 sflvkkksns isvgefyrda vlqrcspnlq rhsnslgpif dhedllkrkr kilssddslr
  961 ssklqshmrh sdsisslase reyitsldls anelrdidal sqkccisvhl ehleklelhq
1021 naltsfpqql cetlkslthl dlhsnkftsf psyllkmsci anldvsrndi gpsvvldptv
1081 kcptlkqfnl synqlsfvpe nltdvvekle qlilegnkis gicsplrlke lkilnlsknh
1141 isslsenfle acpkvesfsa rmnflaampf lppsmtilkl sqnkfscipe ailnlphlrs
1201 ldmssndiqy lpgpahwksl nlrellfshn qisildlsek aylwsrvekl hlshnklkei
1261 ppeigclenl tsldvsynle lrsfpnemgk lskiwdlpld elhlnfdfkh igckakdiir
1321 flqqrlkkav pynrmklmiv gntgsgkttl lqqlmktkks dlgmqsatvg idvkdwpiqi
1381 rdkrkrdlvl nvwdfagree fysthphfmt qralylavyd lskgqaevda mkpwlfnika
1441 rassspvilv gthldvsdek qrkacmskit kellnkrgfp airdyhfvna teesdalakl
1501 rktiinesln fkirdqlvvg glipdcyvel ekiilserkn vpiefpvidr krllqlvren
1561 qlqldenelp havhflnesg vllhfqdpal qlsdlyfvep kwlckimagi ltvkvegcpk
1621 hpkgiisrrd vekflskkrk fpknymsqyf kllekfqial pigeeyllvp sslsdhrpvi
1681 elphcensei iirlyempyf pmgfwsrlin rlleispyml sgreralrpn rmywrqgiyl
1741 nwspeayclv gsevldnhpe sflkitvpsc rkgcillgqv vdhidslmee wfpglleidi
1801 cgegetllkk walysfndge ehqkillddl mkkaeegdll vnpdqprlti pisqiapdli
1861 ladlprniml nndelefeqa pefllgdgsf gsvyraayeg eevavkifnk htslrllrqe
1921 lvvlchlhhp slisllaagi rprmlvmela skgsldrllq qdkasltrtl qhrialhvad
1981 glrylhsami iyrdlkphnv llftlypnaa iiakiadygi aqyccrmgik tsegtpgfra
2041 pevargnviy nqqadvysfg lllydilttg griveglkfp nefdeleiqg klpdpvkeyg
2101 capwpmvekl ikqclkenpq erptsaqvfd ilnsaelvcl trrillpknv ivecmvathh
2161 nsrnasiwlg cghtdrgqls fldIntegyt seevadsril clalvhlpve keswivsgtq
2221 sgtllvinte dgkkrhtlek mtdsvtclyc nsfskqskqk nfllvgtadg klaifedktv
2281 klkgaaplki lnignvstpl mclsestnst ernvmwggcg tkifsfsndf tiqklietrt
2341 sqlfsyaafs dsniitvvvd talyiakqns pvvevwdkkt eklcglidcv hflrevmvke
2401 nkeskhkmsy sgrvktlclq kntalwigtg gghillldls trrlirviyn fcnsvrvmmt
2461 aqlgslknvm lvlgynrknt egtqkqkeiq scltvwdinl phevqnlekh ievrkelaek
2521 mrrtsve
```

In some embodiments, the disease or disorder is characterized or mediated by aberrant activity of a LRRK2 mutant.

In some embodiments, the LRRK2 mutant has a G2019S mutation relative to the wild-type protein. In some embodiments, the LRRK2 mutant has a A2016T mutation relative to the wild-type protein. In some embodiments, the LRRK2 mutant has a R1441G mutation relative to the wild-type protein. In some embodiments, the LRRK2 mutant has a G2385R mutation relative to the wild-type protein.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

In some embodiments, the disease or disorder is a non-cancerous disease or disorder.

In some embodiments, the disease or disorder is neurodegenerative disease or disorder.

Exemplary neurodegenerative diseases or disorders that may be amenable to treatment with the compounds of the present disclosure includes Parkinson's disease (PD), Prion disease, Huntington's disease, Alzheimer's disease, multiple system atrophy, Pick's disease, progressive supranuclear palsy (PSP), frontotemporal dementia (FTD), corticobasal degeneration (CBD), chronic traumatic encephalopathy, argyrophilic grains disease, tangle-dominant dementia, and primary age-related tauopathy (PART).

In some embodiments, the neurodegenerative disease is Parkinson's disease (PD).

Other exemplary types of non-cancerous (e.g., cell proliferative) diseases or disorders that may be amenable to treatment with the compounds of the present disclosure include inflammatory diseases and conditions, autoimmune diseases, heart diseases, viral diseases, chronic and acute kidney diseases or injuries, metabolic diseases, and allergic and genetic diseases.

Representative examples of specific non-cancerous diseases and disorders include rheumatoid arthritis, alopecia areata, lymphoproliferative conditions, autoimmune hematological disorders (e.g. hemolytic anemia, aplastic anemia, anhidrotic ectodermal dysplasia, pure red cell anemia and idiopathic thrombocytopenia), cholecystitis, acromegaly, rheumatoid spondylitis, osteoarthritis, gout, scleroderma, sepsis, septic shock, dacryoadenitis, cryopyrin associated periodic syndrome (CAPS), endotoxic shock, endometritis, gram-negative sepsis, keratoconjunctivitis sicca, toxic shock syndrome, asthma, adult respiratory distress syndrome, chronic obstructive pulmonary disease, chronic pulmonary inflammation, chronic graft rejection, hidradenitis suppurativa, inflammatory bowel disease, Crohn's disease, Behcet's syndrome, systemic lupus erythematosus, glomerulonephritis, multiple sclerosis, juvenile-onset diabetes, autoimmune uveoretinitis, autoimmune vasculitis, thyroiditis, Addison's disease, lichen planus, appendicitis, bullous pemphigus, pemphigus vulgaris, pemphigus foliaceus, paraneoplastic pemphigus, myasthenia gravis, immunoglobulin A nephropathy, Hashimoto's disease, Sjogren's syndrome, vitiligo, Wegener granulomatosis, granulomatous orchitis, autoimmune oophoritis, sarcoidosis, rheumatic carditis, ankylosing spondylitis, Grave's disease, autoimmune thrombocytopenia purpura, psoriasis, psoriatic arthritis, eczema, dermatitis herpetiformis, ulcerative colitis, pancreatic fibrosis, hepatitis, hepatic fibrosis, CD14 mediated sepsis, non-CD14 mediated sepsis, acute and chronic renal disease, irritable bowel syndrome, pyresis, restenosis, cervicitis, stroke and ischemic injury, neural trauma, acute and chronic pain, allergic rhinitis, allergic conjunctivitis, chronic heart failure, congestive heart failure, acute coronary syndrome, cachexia, malaria, leprosy, leishmaniosis, Lyme disease, Reiter's syndrome, acute synovitis, muscle degeneration, bursitis, tendonitis, tenosynovitis, herniated, ruptured, or prolapsed intervertebral disk syndrome, osteopetrosis, rhinosinusitis, thrombosis, silicosis, pulmonary sarcosis, bone resorption diseases, such as osteoporosis, fibromyalgia, AIDS and other viral diseases such as Herpes Zoster, Herpes Simplex I or II, influenza virus and cytomegalovirus, diabetes Type I and II, obesity, insulin resistance and diabetic retinopathy, 22q11.2 deletion syndrome, Angelman syndrome, Canavan disease, celiac disease, Charcot-Marie-Tooth disease, color blindness, Cri du chat, Down syndrome, cystic fibrosis, Duchenne muscular dystrophy, haemophilia, Klinefleter's syndrome, neurofibromatosis, phenylketonuria, Prader-Willi syndrome, sickle cell disease, Tay-Sachs disease, Turner syndrome, urea cycle disorders, thalassemia, otitis, pancreatitis, parotitis, pericarditis, peritonitis, pharyngitis, pleuritis, phlebitis, pneumonitis, uveitis, polymyositis, proctitis, interstitial lung fibrosis, dermatomyositis, atherosclerosis, arteriosclerosis, amyotrophic lateral sclerosis, asociality, varicosis, vaginitis, depression, Sudden Infant Death Syndrome, and non-cancerous meningioma.

In other embodiments, the methods are directed to treating subjects having cancer. Generally, the compounds of the present disclosure may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer (e.g., urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), polycythemia vera, lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), germ cell tumor, ovarian germ cell tumor, head and neck cancer, Hodgkin's lymphoma, leukemia, lymphoma, multiple myeloma, hepatocellular carcinoma, hypopharyngeal cancer, intraocular melanoma, ocular cancer, islet cell tumors (endocrine pancreas), renal cancer (e.g., Wilm's Tumor, clear cell renal cell carcinoma), liver cancer, lung cancer (e.g., non-small cell lung cancer and small cell lung cancer), Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, essential thrombocythemia, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, pituitary tumor, plasma cell neoplasm, pleuropulmonary blastoma, prostate cancer, retinoblastoma rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, transitional cell cancer of the renal pelvis and ureter and other urinary organs, urethral cancer, gestational trophoblastic tumor, vaginal cancer vulvar cancer and cancerous meningioma.

Sarcomas that may be treatable with compounds of the present disclosure include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types).

In some embodiments, methods of the present disclosure entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematological system” include lymphoma, leukemia, myeloid neoplasms, mast cell neoplasms, myelodysplasia, benign monoclonal gammopathy, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain. In some embodiments, the brain cancer is a glioma or glioblastoma.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SCLC”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, compounds of the present disclosure may be used to treat non-metastatic or metastatic lung cancer (e.g., NSCLC, ALK-positive NSCLC, NSCLC harboring ROSI Rearrangement, Lung Adenocarcinoma, and Squamous Cell Lung Carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell types).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

The bifunctional compounds of the present disclosure and their pharmaceutically acceptable salts and stereoisomers may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been unsuccessful, or partially successful but who became non-responsive or intolerant to the particular treatment. Therapy may also be given as an adjuvant treatment, i.e., to prevent reoccurrence of cancer in patients with no currently detectable disease or after surgical removal of a tumor. Thus, in some embodiments, the compound may be administered to a patient who has received prior therapy, such as chemotherapy, radioimmunotherapy, surgical therapy, immunotherapy, radiation therapy, targeted therapy or any combination thereof.

The methods of the present disclosure may entail administration of a bifunctional compound or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day off period. In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of five days.

### Combination Therapy

The bifunctional compounds of the present disclosure and their pharmaceutically acceptable salts or stereoisomers may be used in combination or concurrently with at least one other active agent e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second agent, the first of the two agents is in some cases still detectable at effective concentrations at the site of treatment. The sequence and time interval may be determined such that they can act together (e.g., synergistically to provide an increased benefit than if they were administered otherwise). For example, the agents may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

The dosage of the additional, e.g., anticancer, therapeutic may be the same or even lower than known or recommended doses. See, Hardman et al., eds., Goodman & Gilman's The Pharmacological Basis of Therapeutics, 10th ed., McGraw-Hill, New York, 2001; Physician's Desk Reference 60th ed., 2006.

In some embodiments, a bifunctional compound of the disclosure and the additional anticancer therapeutic agent may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more anticancer therapeutics may be administered within the same patient visit.

In some embodiments, a bifunctional compound of the present disclosure and the additional therapeutic agent (e.g., an anti-cancer therapeutic) are cyclically administered. By way of example in the context of cancer treatment, cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, a bifunctional compound of the present disclosure may be used in combination with one or more of Levodopa, Sinemet, Safinamide, Ropinirole, Pramipexole, Rotigotine Amantadine, Artane, Cogentin, Eldepryl, Zelapar, and Azilect (e.g., for Parkinson's disease). In some embodiments, a bifunctional compound of the present disclosure may be used in combination with one or more of Aricept, Exelon, Razadyne, Namenda, and Namzaric (e.g., for Alzheimer's disease). In some embodiments, a bifunctional compound of the present disclosure may be used in combination with one or more of Xenazine, Haldol, chlorpromazine, Risperdal, Seroquel, Keppra, Klonopin, Celexa, Prozac, Epitol, and Depacon (e.g., for Huntington's disease). In some embodiments, a bifunctional compound of the present disclosure may be used in combination with one or more of trazodone, Zoloft, Luvox, Zyprexa, and Seroquel (e.g., for Pick's syndrome).

Representative examples of other active agents known to treat neurodegenerative diseases and disorders, and which may be used in conjunction with the bifunctional compounds, include dopaminergic treatments (e.g., Carbidopa-levodopa, pramipexole (Mirapex), ropinirole (Requip) and rotigotine (Neupro, given as a patch)). Apomorphine and monoamine oxidase B (MAO-B) inhibitors (e.g., selegiline (Eldepryl, Zelapar), rasagiline (Azilect) and safinamide (Xadago)) for PD and movement disorders, cholinesterase inhibitors for cognitive disorders (e.g., benztropine (Cogentin) or trihexyphenidyl), antipsychotic drugs for behavioral and psychological symptoms of dementia, as well as agents aimed to slow the development of diseases, such as Riluzole for ALS, cerebellar ataxia and Huntington's disease, non-steroidal anti-inflammatory drugs for Alzheimer's disease, and caffeine A2A receptor antagonists and CERE-120 (adeno-associated virus serotype 2-neurturin) for the neuroprotection of Parkinson's disease.

Representative types of additional anti-cancer agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immune-modulators, therapeutic antibodies (e.g., mono-specific and bispecific antibodies) and CAR-T therapy.

Examples of anti-cancer agents that may be used in combination with the bifunctional compounds are known in the art. See, e.g., U.S. Pat. No. 9,101,622 (Section 5.2 thereof) and U.S. Pat. No. 9,345,705 B2 (Columns 12-18 thereof). In some embodiments, a bifunctional compound of the present disclosure may be used in combination with at least one other anti-cancer agent such as Paclitaxel (e.g., ovarian cancer, breast cancer, lung cancer, Kaposi sarcoma, cervical cancer, and pancreatic cancer), Topotecan (e.g., ovarian cancer and lung cancer), Irinotecan (e.g., colon cancer, and small cell lung cancer), Etoposide (e.g., testicular cancer, lung cancer, lymphomas, and non-lymphocytic leukemia), Vincristine (e.g., leukemia), Leucovorin (e.g., colon cancer), Altretamine (e.g., ovarian cancer), Daunorubicin (e.g., acute myeloid leukemia (AML), acute lymphocytic leukemia (ALL), chronic myelogenous leukemia (CML), and Kaposi's sarcoma), Trastuzumab (e.g., breast cancer, stomach cancer, and esophageal cancer), Rituximab (e.g., non-Hodgkin's lymphoma), Cetuximab (e.g., colorectal cancer, metastatic non-small cell lung cancer and head and neck cancer), Pertuzumab (e.g., metastatic HER2-positive breast cancer), Alemtuzumab (e.g., chronic lymphocytic leukemia (CLL), cutaneous T-cell lymphoma (CTCL) and T-cell lymphoma), Panitumumab (e.g., colon and rectum cancer), Tamoxifen (e.g., breast cancer), Fulvestrant (e.g., breast cancer), Letrazole (e.g., breast cancer), Exemestane (e.g., breast cancer), Azacytidine (e.g., myelodysplastic syndromes), Mitomycin C (e.g., gastro-intestinal cancers, anal cancers, and breast cancers), Dactinomycin (e.g., Wilms tumor, rhabdomyosarcoma, Ewing's sarcoma, trophoblastic neoplasm, testicular cancer, and ovarian cancer), Erlotinib (e.g., non-small cell lung cancer and pancreatic cancer), Sorafenib (e.g., kidney cancer and liver cancer), Temsirolimus (e.g., kidney cancer), Bortezomib (e.g., multiple myeloma and mantle cell lymphoma), Pegaspargase (e.g., acute lymphoblastic leukemia), Cabometyx (e.g., hepatocellular carcinoma, medullary thyroid cancer, and renal cell carcinoma), Keytruda (e.g., cervical cancer, gastric cancer, hepatocellular carcinoma, Hodgkin's lymphoma, melanoma, Merkel cell carcinoma, non-small cell lung cancer, urothelial carcinoma, and squamous cell carcinoma of the head and neck), Nivolumab (e.g., colorectal cancer, hepatocellular carcinoma, melanoma, non-small cell lung cancer, renal cell carcinoma, small cell lung cancer, and urothelial carcinoma), and Regorafenib (e.g., colorectal cancer, gastrointestinal stromal tumor, and hepatocellular carcinoma).

### Pharmaceutical Kits

The present compositions may be assembled into kits or pharmaceutical systems. Kits or pharmaceutical systems according to this aspect of the disclosure include a carrier or package such as a box, carton, tube or the like, having in close confinement therein one or more containers, such as vials, tubes, ampoules, or bottles, which contain a compound of the present disclosure or a pharmaceutical composition which contains the compound and a pharmaceutically acceptable carrier wherein the compound and the carrier may be disposed in the same or separate containers. The kits or pharmaceutical systems of the disclosure may also include printed instructions for using the compounds and compositions.

### EXAMPLES

These and other aspects of the present disclosure will be further appreciated upon consideration of the following Examples, which are intended to illustrate certain particular embodiments of the disclosure but are not intended to limit its scope, as defined by the claims.

### General Methods

Unless indicated otherwise, reagents and solvents were used as received from commercial suppliers. All reactions were monitored using a Waters® Acquity ultra performance liquid chromatography/mass spectrometry (UPLC/MS) system using Acquity UPLC® BEH C18 column (2.1×50 mm, 1.7 μm particle size). UPLC method A: solvent gradient=90% A at 0 min, 5% A at 1.8 min; method B: solvent gradient=85% A at 0 min, 1% A at 1.8 min; solvent A=0.1% formic acid in H₂O; solvent B=0.1% formic acid in acetonitrile; flow rate: 0.6 mL/min. Purification of reaction products was carried out by flash chromatography using CombiFlash® Rf with Teledyne ISCO RediSep® normal-phase silica flash columns; or Waters® high performance liquid chromatography (HPLC) system using SunFire™ C18 column (19×100 mm, 5 μm particle size): solvent gradient 0% to 99% acetonitrile in H₂O (0.035% trifluoroacetic acid (TFA) as additive); flow rate: 20 mL/min, or SunFire™ C18 column (30×250 mm, 5 μm particle size): solvent gradient 0% to 99% acetonitrile in H₂O (0.035% TFA as additive); flow rate: 40 mL/min. The purity of all compounds was over 95% and was analyzed with Waters® UPLC system. ¹H NMR and ¹³C NMR spectra were obtained using Bruker Avance III spectrometers (500 MHz for ¹H, and 125 MHz for ¹³C). Chemical shifts are reported relative to deuterated methanol (δ=3.31) or dimethyl sulfoxide (DMSO) (δ=2.50) for ¹H NMR. Spectra are given in ppm (δ) and as br=broad, s=singlet, d=doublet, t=triplet, q=quartet, m=multiplet and coupling constants (J) are reported in Hertz.

### Example 1: Synthesis of (1)

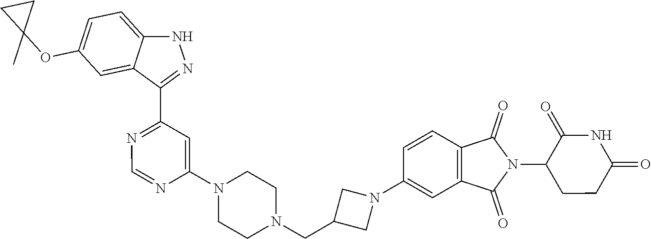 [SMILES: CC1(Oc2ccc3[nH]nc(-c4cc(N5CCN(CC6CN(c7ccc8c(c7)C(=O)N(C7CCC(=O)NC7=O)C8=O)C6)CC5)ncn4)c3c2)CC1]

### 2-(2,6-dioxopiperidin-3-yl)-5-(3-((4-(6-(5-(1-methylcyclopropoxy)-1H-indazol-3-yl)pyrimidin-4-yl)piperazin-1-yl)methyl)azetidin-1-yl)isoindoline-1,3-dione (1)

The title compound was prepared according to the following synthetic scheme.

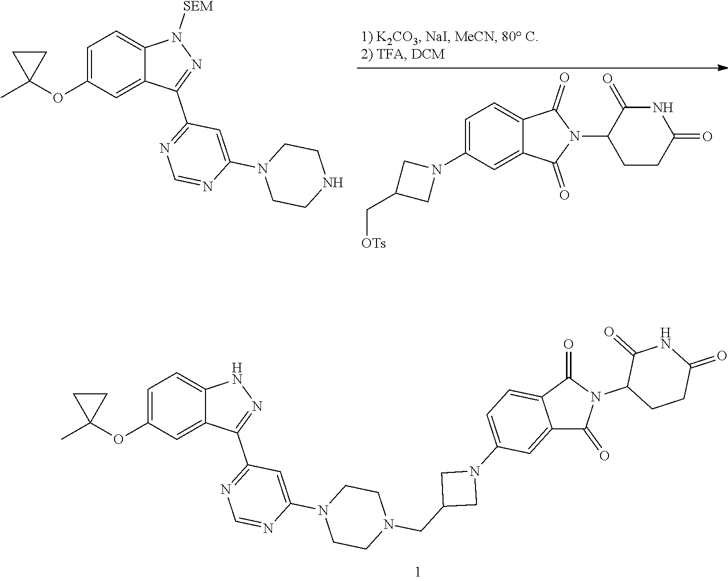 [SMILES: CC1(Oc2ccc3c(c2)c(-c2cc(N4CCNCC4)ncn2)nn3COCC[Si](C)(C)C)CC1 ; Cc1ccc(S(=O)(=O)OCC2CN(c3ccc4c(c3)C(=O)N(C3CCC(=O)NC3=O)C4=O)C2)cc1 ; CC1(Oc2ccc3[nH]nc(-c4cc(N5CCN(CC6CN(c7ccc8c(c7)C(=O)N(C7CCC(=O)NC7=O)C8=O)C6)CC5)ncn4)c3c2)CC1]

To a solution of 5-(1-methylcyclopropoxy)-3-(6-(piperazin-1-yl)pyrimidin-4-yl)-1-((2-(trimethylsilyl)ethoxy)methyl)-1H-indazole (20 mg, 0.042 mmol), prepared according to the procedure found in International Application Publication No. WO 2020/081682, and (1-(2-(2,6-dioxopiperidin-3-yl)-1,3-dioxoisoindolin-5-yl)azetidin-3-yl)methyl 4-methylbenzenesulfonate (21 mg, 0.042 mmol), prepared according to the procedure found in International Application Publication No. WO 2020/038415, in MeCN (5 mL) was added NaI (32 mg, 0.21 mmol) and K₂CO₃ (12 mg, 0.08 mmol). The reaction mixture was stirred at 100° C. for 2 hr. 1-(2-(2,6-dioxopiperidin-3-yl)-1,3-dioxoisoindolin-5-yl)azetidin-3-yl)methyl 4-methylbenzenesulfonate (10 mg, 0.021 mmol) was added and the mixture stirred another 1 hour. The mixture was cooled to rt, quenched with H₂O and extracted with EtOAc. The combined organic layer was washed with brine, dried over MgSO₄ and condensed to give a brown oil that was dissolved in CH₂Cl₂ (10 mL). TFA (1 mL) was added and the mixture stirred for 1 hr. The solvent was removed, and the residue purified by reversed phase HPLC using a gradient of 0-70% MeCN in H₂O to give the desired product as a yellow solid ((11 mg, 39% yield) ESI m/z: 676.43).

### Example 2: Synthesis of (2)

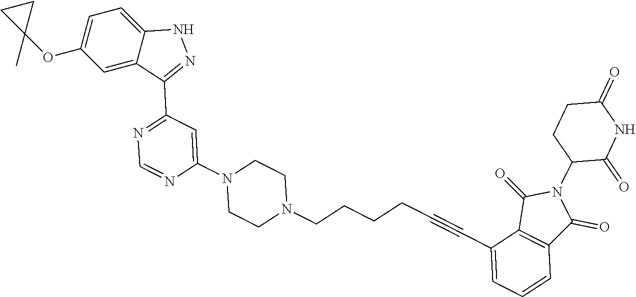 [SMILES: CC1(Oc2ccc3[nH]nc(-c4cc(N5CCN(CCCCC#Cc6cccc7c6C(=O)N(C6CCC(=O)NC6=O)C7=O)CC5)ncn4)c3c2)CC1]

### 2-(2,6-dioxopiperidin-3-yl)-4-(6-(4-(6-(5-(1-methylcyclopropoxy)-1H-indazol-3-yl)pyrimidin-4-yl)piperazin-1-yl)hex-1-yn-1-yl)isoindoline-1,3-dione (2)

The title compound was prepared according to the following synthetic scheme.

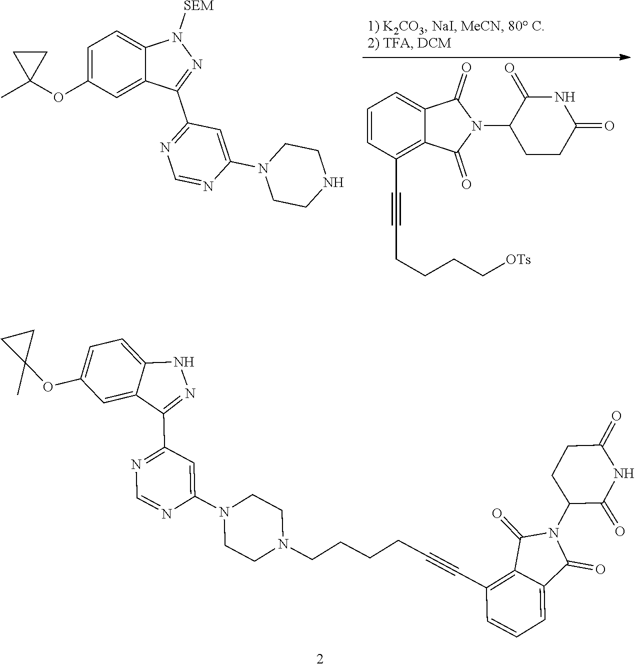 [SMILES: CC1(Oc2ccc3c(c2)c(-c2cc(N4CCNCC4)ncn2)nn3COCC[Si](C)(C)C)CC1 ; Cc1ccc(S(=O)(=O)OCCCCC#Cc2cccc3c2C(=O)N(C2CCC(=O)NC2=O)C3=O)cc1 ; CC1(Oc2ccc3[nH]nc(-c4cc(N5CCN(CCCCC#Cc6cccc7c6C(=O)N(C6CCC(=O)NC6=O)C7=O)CC5)ncn4)c3c2)CC1]

The title compound was prepared using the procedure of Example 1 using 6-(2-(2,6-dioxopiperidin-3-yl)-1,3-dioxoisoindolin-4-yl)hex-5-yn-1-yl 4-methylbenzenesulfonate (42 mg, 0.08 mmol), prepared according to Su, S., et al., J. Med. Chem. 62 (16): 7575-7582, (2019) to give a yellow solid ((18 mg, 33% yield) ESI m/z: 687.62).

### Example 3: Synthesis of (3)

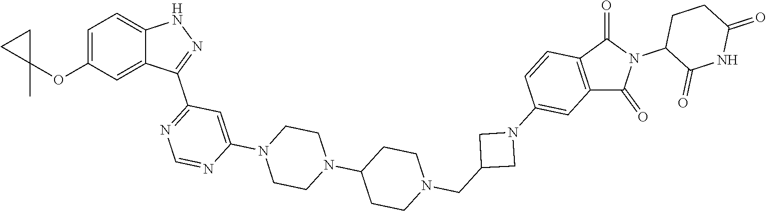 [SMILES: CC1(Oc2ccc3[nH]nc(-c4cc(N5CCN(C6CCN(CC7CN(c8ccc9c(c8)C(=O)N(C8CCC(=O)NC8=O)C9=O)C7)CC6)CC5)ncn4)c3c2)CC1]

### 2-(2,6-dioxopiperidin-3-yl)-5-(3-((4-(4-(6-(5-(1-methylcyclopropoxy)-1H-indazol-3-yl)pyrimidin-4-yl)piperazin-1-yl)piperidin-1-yl)methyl)azetidin-1-yl)isoindoline-1,3-dione (3)

### Synthesis of 2-(2,6-dioxopiperidin-3-yl)-5-(3-((4-oxopiperidin-1-yl)methyl)azetidin-1-yl)isoindoline-1,3-dione

The title compound was prepared according to the following synthetic scheme.

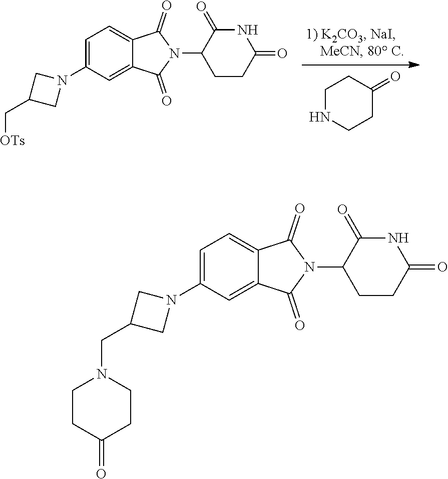 [SMILES: Cc1ccc(S(=O)(=O)OCC2CN(c3ccc4c(c3)C(=O)N(C3CCC(=O)NC3=O)C4=O)C2)cc1 ; O=C1CCNCC1 ; O=C1CCN(CC2CN(c3ccc4c(c3)C(=O)N(C3CCC(=O)NC3=O)C4=O)C2)CC1]

To a solution (1-(2-(2,6-dioxopiperidin-3-yl)-1,3-dioxoisoindolin-5-yl)azetidin-3-yl)methyl 4-methylbenzenesulfonate (200 mg, 0.41 mmol) and piperidin-4-one (56 mg, 0.41 mmol) in MeCN (15 mL) was added NaI (12 mg, 0.02 mmol) and K₂CO₃ (278 mg, 2.01 mmol). The reaction mixture was stirred at rt for 2 hr. The mixture was cooled to rt, quenched with H₂O, and extracted with EtOAc. The combined organic layer was washed with brine, dried over MgSO₄. and condensed to give a brown oil that was used without further purification (ESI m/z: 426.73).

### Synthesis of 2-(2,6-dioxopiperidin-3-yl)-5-(3-((4-(4-(6-(5-(1-methylcyclopropoxy)-1H-indazol-3-yl)pyrimidin-4-yl)piperazin-1-yl)piperidin-1-yl)methyl)azetidin-1-yl)isoindoline-1,3-dione (3)

The title compound was prepared according to the following synthetic scheme.

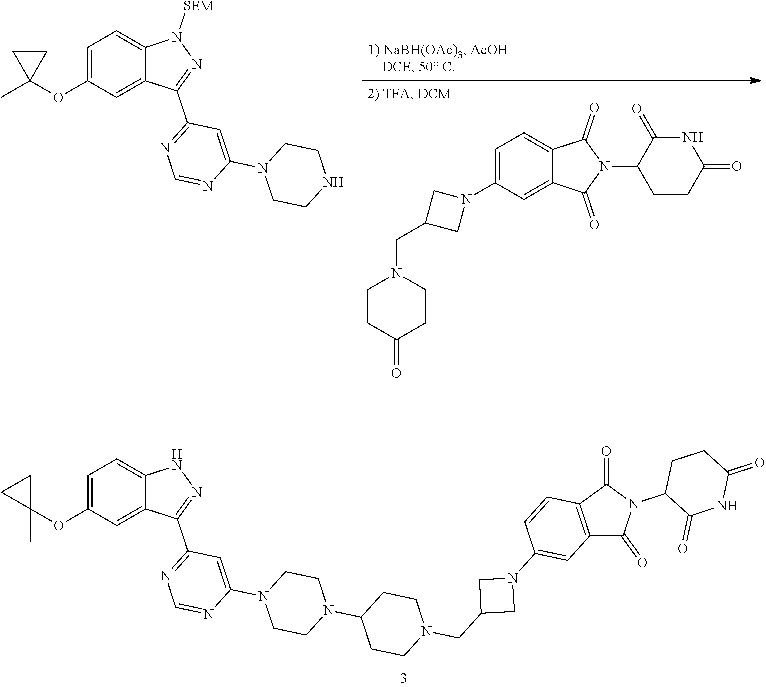 [SMILES: CC1(Oc2ccc3c(c2)c(-c2cc(N4CCNCC4)ncn2)nn3COCC[Si](C)(C)C)CC1 ; O=C1CCN(CC2CN(c3ccc4c(c3)C(=O)N(C3CCC(=O)NC3=O)C4=O)C2)CC1 ; CC1(Oc2ccc3[nH]nc(-c4cc(N5CCN(C6CCN(CC7CN(c8ccc9c(c8)C(=O)N(C8CCC(=O)NC8=O)C9=O)C7)CC6)CC5)ncn4)c3c2)CC1]

To a solution of 5-(1-methylcyclopropoxy)-3-(6-(piperazin-1-yl)pyrimidin-4-yl)-1-((2-(trimethylsilyl)ethoxy)methyl)-1H-indazole (21 mg, 0.043 mmol) in DCE (5 mL) was added 2-(2,6-dioxopiperidin-3-yl)-5-(3-((4-oxopiperidin-1-yl)methyl)azetidin-1-yl)isoindoline-1,3-dione (20 mg, 0.047 mmol) followed by NaBH(OAc)₃ (91 mg, 0.43 mmol) and AcOH (10 drops). The mixture was stirred at 50° C. for 8 hrs. The mixture was cooled to rt, quenched with H₂O, and extracted with EtOAc. The combined organic layer was washed with brine, dried over MgSO₄, and condensed to give a brown oil that was dissolved in CH₂Cl₂ (10 mL). TFA (1 mL) was added and the mixture stirred for 1 hr. The solvent was removed, and the residue purified by reversed phase HPLC using a gradient of 0-70% MeCN in H₂O to give the desired product as a yellow solid ((7 mg, 20% yield) ESI m/z: 759.52).

### Example 4: Synthesis of (4)

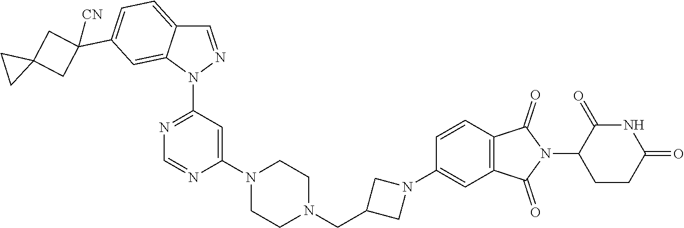 [SMILES: N#CC1(c2ccc3cnn(-c4cc(N5CCN(CC6CN(c7ccc8c(c7)C(=O)N(C7CCC(=O)NC7=O)C8=O)C6)CC5)ncn4)c3c2)CC2(CC2)C1]

### 5-(1-(6-(4-((1-(2-(2,6-dioxopiperidin-3-yl)-1,3-dioxoisoindolin-5-yl)azetidin-3-yl)methyl)piperazin-1-yl)pyrimidin-4-yl)-1H-indazol-6-yl)spiro[2.3]hexane-5-carbonitrile (4)

The title compound was prepared according to the following synthetic scheme.

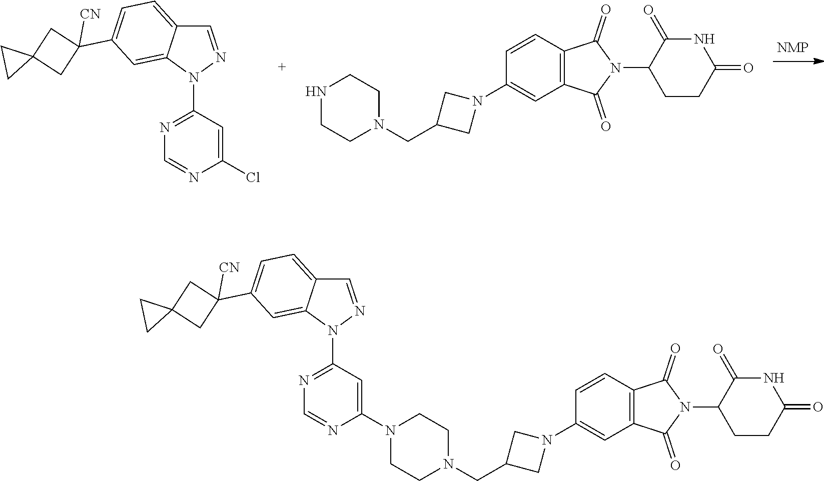 [SMILES: N#CC1(c2ccc3cnn(-c4cc(Cl)ncn4)c3c2)CC2(CC2)C1 ; O=C1CCC(N2C(=O)c3ccc(N4CC(CN5CCNCC5)C4)cc3C2=O)C(=O)N1 ; N#CC1(c2ccc3cnn(-c4cc(N5CCN(CC6CN(c7ccc8c(c7)C(=O)N(C7CCC(=O)NC7=O)C8=O)C6)CC5)ncn4)c3c2)CC2(CC2)C1]

A solution of 5-(1-(6-chloropyrimidin-4-yl)-1H-indazol-6-yl)spiro[2.3]hexane-5-carbonitrile (6.7 mg, 0.020 mmol, 1.0 equiv.), prepared according to the method described in WO 2019074810, and 2-(2,6-dioxopiperidin-3-yl)-5-(3-(piperazin-1-ylmethyl)azetidin-1-yl)isoindoline-1,3-dione (8.2 mg, 0.020 mmol, 1.0 equiv.) in NMP (0.2 mL) was heated to 130° C. and stirred for 2 h. After cooling to ambient temperature, the reaction mixture was purified by preparative HPLC (t_{R}=27.9 min) and lyophilized to afford the desired product as a yellow solid ((7.0 mg, 50% yield). LCMS C₃₉H₃₉N₁₀O₄ (M+H)⁺711.45, t_{R}=1.07 min).

### Example 5: Synthesis of (5)

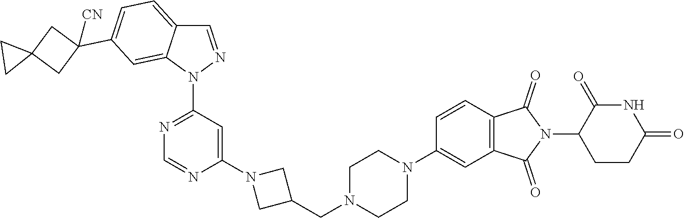 [SMILES: N#CC1(c2ccc3cnn(-c4cc(N5CC(CN6CCN(c7ccc8c(c7)C(=O)N(C7CCC(=O)NC7=O)C8=O)CC6)C5)ncn4)c3c2)CC2(CC2)C1]

### 5-(1-(6-(3-((4-(2-(2,6-dioxopiperidin-3-yl)-1,3-dioxoisoindolin-5-yl)piperazin-1-yl)methyl)azetidin-1-yl)pyrimidin-4-yl)-1H-indazol-6-yl)spiro[2.3]hexane-5-carbonitrile (5)

The title compound was prepared according to the following synthetic scheme.

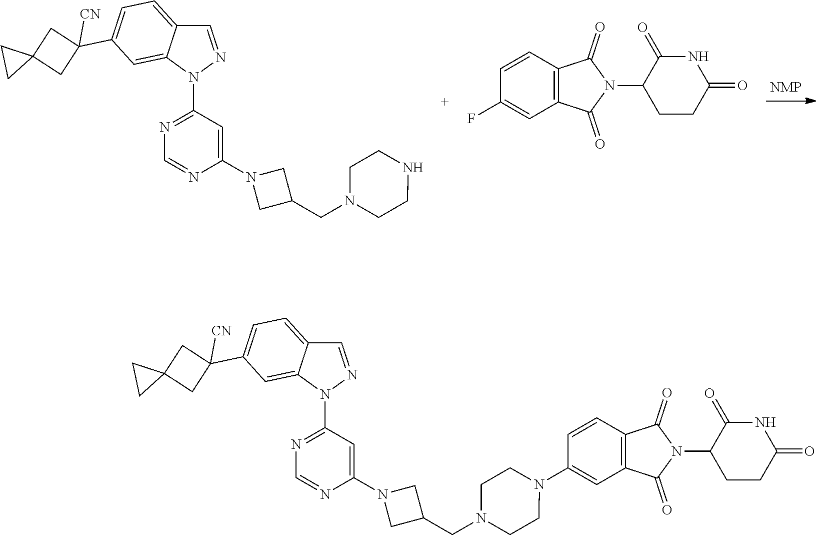 [SMILES: N#CC1(c2ccc3cnn(-c4cc(N5CC(CN6CCNCC6)C5)ncn4)c3c2)CC2(CC2)C1 ; O=C1CCC(N2C(=O)c3ccc(F)cc3C2=O)C(=O)N1 ; N#CC1(c2ccc3cnn(-c4cc(N5CC(CN6CCN(c7ccc8c(c7)C(=O)N(C7CCC(=O)NC7=O)C8=O)CC6)C5)ncn4)c3c2)CC2(CC2)C1]

A solution of 5-(1-(6-(3-(piperazin-1-ylmethyl)azetidin-1-yl)pyrimidin-4-yl)-1H-indazol-6-yl)spiro[2.3]hexane-5-carbonitrile (8.2 mg, 0.018 mmol, 1.0 equiv.) and 2-(2,6-dioxopiperidin-3-yl)-5-fluoroisoindoline-1,3-dione (5.0 mg, 0.020 mmol, 1.0 equiv.) in NMP (0.2 mL) was heated to 130° C. and stirred for 2 h. After cooling to ambient temperature, the reaction mixture was purified by HPLC (t_{R}=27.1 min) to afford the desired product as a brownish solid after lyophilization ((2.0 mg, 15% yield). LCMS C₃₉H₃₉N₁₀O₄ (M+H)⁺711.34, t_{R}=1.05 min).

### Example 6: Synthesis of (6)

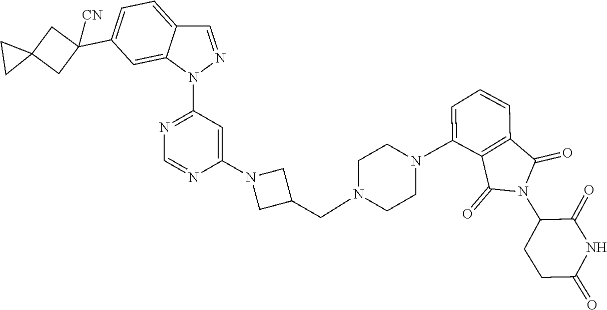 [SMILES: N#CC1(c2ccc3cnn(-c4cc(N5CC(CN6CCN(c7cccc8c7C(=O)N(C7CCC(=O)NC7=O)C8=O)CC6)C5)ncn4)c3c2)CC2(CC2)C1]

5-(1-(6-(3-((4-(2-(2,6-dioxopiperidin-3-yl)-1,3-dioxoisoindolin-4-yl)piperazin-1-yl)methyl)azetidin-1-yl)pyrimidin-4-yl)-1H-indazol-6-yl)spiro[2.3]hexane-5-carbonitrile (6)

The title compound was prepared according to the following synthetic scheme.

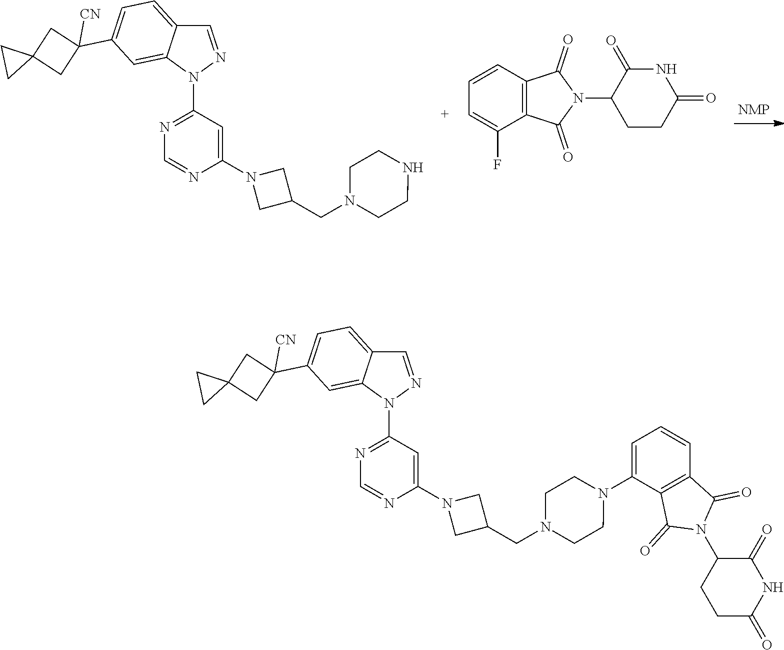 [SMILES: N#CC1(c2ccc3cnn(-c4cc(N5CC(CN6CCNCC6)C5)ncn4)c3c2)CC2(CC2)C1 ; O=C1CCC(N2C(=O)c3cccc(F)c3C2=O)C(=O)N1 ; N#CC1(c2ccc3cnn(-c4cc(N5CC(CN6CCN(c7cccc8c7C(=O)N(C7CCC(=O)NC7=O)C8=O)CC6)C5)ncn4)c3c2)CC2(CC2)C1]

A solution of 5-(1-(6-(3-(piperazin-1-ylmethyl)azetidin-1-yl)pyrimidin-4-yl)-1H-indazol-6-yl)spiro[2.3]hexane-5-carbonitrile (8.2 mg, 0.018 mmol, 1.0 equiv.) and 2-(2,6-dioxopiperidin-3-yl)-4-fluoroisoindoline-1,3-dione (5.0 mg, 0.020 mmol, 1.0 equiv.) in NMP (0.2 mL) was heated to 130° C. and stirred for 2 h. After cooling to ambient temperature, the reaction mixture was purified by HPLC (t_{R}=27.4 min) to afford the desired product as a brownish solid after lyophilization ((3.0 mg, 23% yield). LCMS C₃₉H₃₉N₁₀O₄ (M+H)⁺711.35, t_{R}=1.04 min).

### Example 7: Degradation of LRRK2

Cell lines: Mouse embryonic fibroblast (MEF) WT, LRRK2 homozygous knock-ins in MEFs [R1441C; VPS35N(D620N); G2019S].

Tested concentrations of LRRK2 degraders: 0 nM; 10 nM; 30 nM; 100 nM; 300 nM; 1000 nM.

Experimental time points: 1 h; 6 h; 24 h and 48 h.

Complete growth medium: DMEM supplemented with 10% Fetal Bovine Serum; 1% pen/strep; 1% L-Glutamine; 1% MEM Non-essential Amino Acid Solution; 1% sodium pyruvate.

### List of Commercial and In-House Purified Antibodies

Mouse anti-LRRK2/Dardarin antibody from Antibodies, Inc. (Cat #75-253).

Rabbit monoclonal antibodies for total LRRK2 (UDD3) and pS935-LRRK2 (UDD2) were purified at the University of Dundee (as described in Dzamko et al., PLOS ONE 7, e39132 10.1371/journal.pone.0039132 (2012)).

Loading controls: anti-α-tubulin (Cell Signaling Technology #5174); anti-GAPDH (Santa Cruz Biotechnology Cat. #sc-32233).

(p)Rab10 antibodies: rabbit anti-RAB10 (phospho T73) antibody [MJF-R21] (ab230261); mouse MJFF-total Rab10 monoclonal antibody were generated by nanoTools (nanotools.de); rabbit Rab10 total was from Cell Signaling Technology (Rab10 (D36C4) XP® Rabbit mAb #8127).

Treatment: WT MEF, and cells containing mutant LRRK2 (R1441C, VPS35N and G2019S) cells were plated at equal density into 6-well plates in a final volume of 3 mL of complete growth medium/well. Degraders were reconstituted in DMSO and used at 1:1000 in cells i.e., 3 μL/3 mL. Treatment began when cells were >60% confluent, starting from the longest, 48 h time point, followed by 24 h, 6 h and finally 1 h.

Cell lysis: Media were aspirated, plates were placed on ice and cells were washed with DPBS. Fifty (50) microliters of an ice-cold lysis buffer containing 50 mM Tris-HCl, pH 7.5, 1% (v/v) Triton X-100, 1 mM EGTA, 1 mM sodium orthovanadate, 50 mM NaF, 0.1% (v/v) 2-mercaptoethanol, 10 mM 2-glycerophosphate, 5 mM sodium pyrophosphate, 0.1 μg/mL microcystin-LR (Enzo Life Sciences), 270 mM sucrose and complete EDTA-free protease inhibitor cocktail (Sigma-Aldrich Cat #11836170001) were added per well. Lysates were centrifuged at 20 817 g (14,000 rpm) for 15 min at 4° C. and supernatants were used to determine protein concentration using Bradford assay (Pierce™ Coomassie (Bradford) Protein Assay Kit, Thermo Scientific Cat #23200) and for Western blot analysis.

Western blot analysis: Cell lysates were mixed with 4× SDS-PAGE sample buffer [50 mM Tris —HCl, pH 6.8, 2% (w/v) SDS, 10% (v/v) glycerol, 0.02% (w/v) Bromophenol Blue and 1% (v/v) 2-mercaptoethanol] to a final total protein concentration of 1 μg/μL and heated at 95° C. for 5 min. Twenty (20) micrograms of samples were loaded onto NuPAGE 4-12% Bis-Tris gradient gels (Life Technologies) along with 3 μL of BIO-RAD protein marker (Precision Plus Protein™ All Blue Prestained Protein Standards #1610373kDa), and gels were run in duplicates at 110V for 2 h 30 min with the NuPAGE MOPS SDS running buffer (Life Technologies, Cat #NP0001-02). After electrophoresis, the separated proteins were transferred onto the nitrocellulose membrane (GE Healthcare, Amersham Protran 0.45 μm NC) at 90 V for 90 min. Transferred membranes were briefly stained with Ponceau S stain and divided into 3 strips, as described earlier in Fan, et al., Biochem. J. 475:23-44 (2018). Briefly, upper strip was cut from the top of the membrane to 75 kDa, middle strip cut was between 75 kDa-30 kDa and bottom strip cut was from 30 kDa-to the bottom of the membrane. Membrane strips were blocked at room temperature with 5% (w/v) dried skimmed milk dissolved in TBS-T [20 mM Tris-HCl, pH 7.5, 150 mM NaCl and 0.1% (v/v) Tween 20] for 1 h, washed four times with ten minutes intervals in TBS-T and incubated with primary antibodies diluted in 5% BSA (bovine serum albumin) in TBS-T overnight at 4° C. Primary antibodies were used as follows: upper strip from one of the membranes was incubated with 1 μg/mL of rabbit anti-LRRK2 pS935 UDD2 antibody combined with mouse anti-LRRK2 C-terminus total antibody, while the second upper strip was incubated with anti-LRRK2 N-terminus total antibody (UDD3) at a final concentration of 100 ng/ml; the middle strips were incubated with rabbit anti-α-tubulin (Cell Signaling Technology #5174) and mouse anti-GAPDH antibody (Santa Cruz Biotechnology #sc-32233) at a final concentration of 50 ng/mL. The bottom strips were blotted with rabbit MJFF-pRAB10 monoclonal antibody multiplexed with mouse MJFF-total Rab10 monoclonal antibody at a final concentration of 0.5 μg/mL for each of the antibody and with the total Rab10 (Rab10 (D36C4) XP® Rabbit mAb #8127 Cell Signaling Technology) at a final concentration of 1 μg/mL (Fan et al., supra., Lis, et al., Biochem. J. 475:1-22 (2018)). Membranes were washed as before and incubated at room temperature for 1 h with anti-rabbit and anti-mouse near-infrared fluorescent IRDye antibodies (LI-COR #925-68070, #925-32211) diluted (1:30 000 and 1:15 000, respectively) in TBS-T. Following incubation in secondary antibodies, membrane strips were washed and signal was developed using the LI-COR Odyssey CLx Western Blot imaging system.

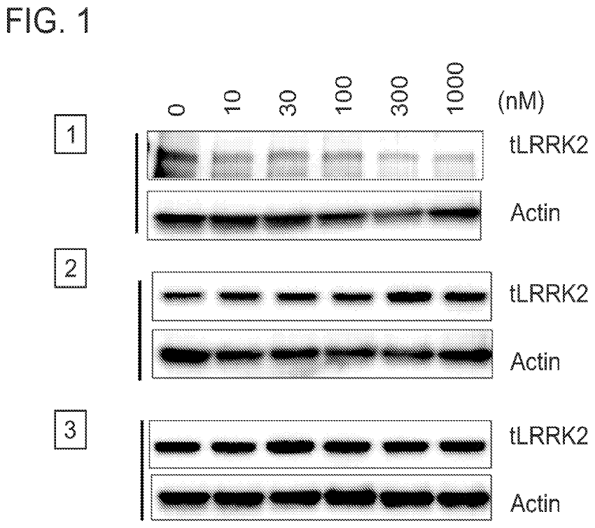

FIG. 1 is a set of immunoblots of 448T cells treated with the indicated compounds 1, 2, and 3 at concentrations ranging from 0 to 1000 nM. The data show that 1 is a potent degrader of LRRK2 with a Dmax of 60% and a DC₅₀ of 33 nM.

All patent publications and non-patent publications are indicative of the level of skill of those skilled in the art to which this disclosure pertains. All these publications are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A bifunctional compound having a structure represented by formula (Ia) or formula (Ib): 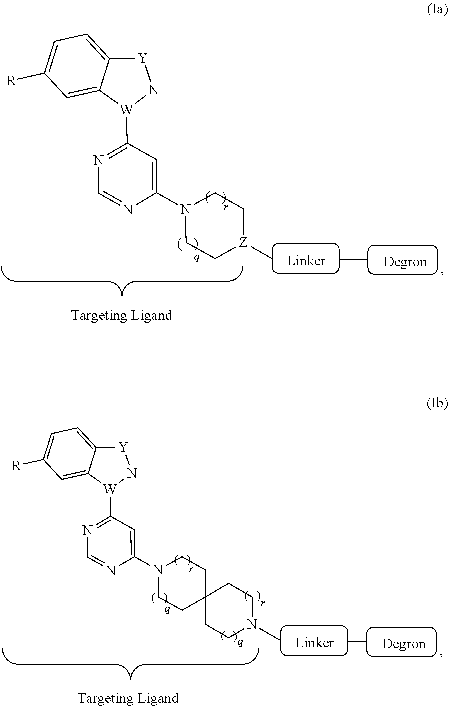
or a pharmaceutically acceptable salt or stereoisomer thereof, wherein
—W—N—Y— is —C═N—NH— or —N—N═CH—;
R is MeO—, EtO—, i-PrO—, 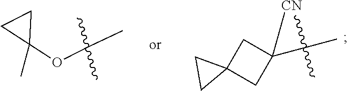
Z is N or CH;
each occurrence of q and r is independently 0 or 1;
the targeting ligand binds leucine-rich repeat kinase 2 (LRRK2);
the degron (“Degron”) represents a moiety that binds an E3 ubiquitin ligase; and
the linker (“Linker”) provides a covalent attachment between the targeting ligand and the degron.

2. The bifunctional compound, or pharmaceutically acceptable salt or stereoisomer thereof of claim 1, wherein the degron binds a Von Hippel-Lindau (VHL) tumor suppressor.

3. The bifunctional compound, or pharmaceutically acceptable salt or stereoisomer thereof of claim 2, wherein the degron is represented by any one of the following structures:

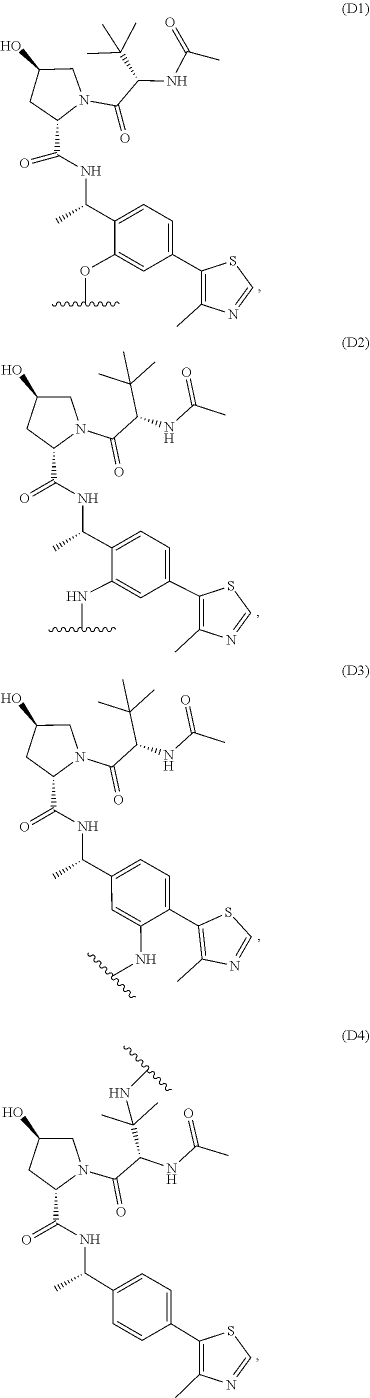

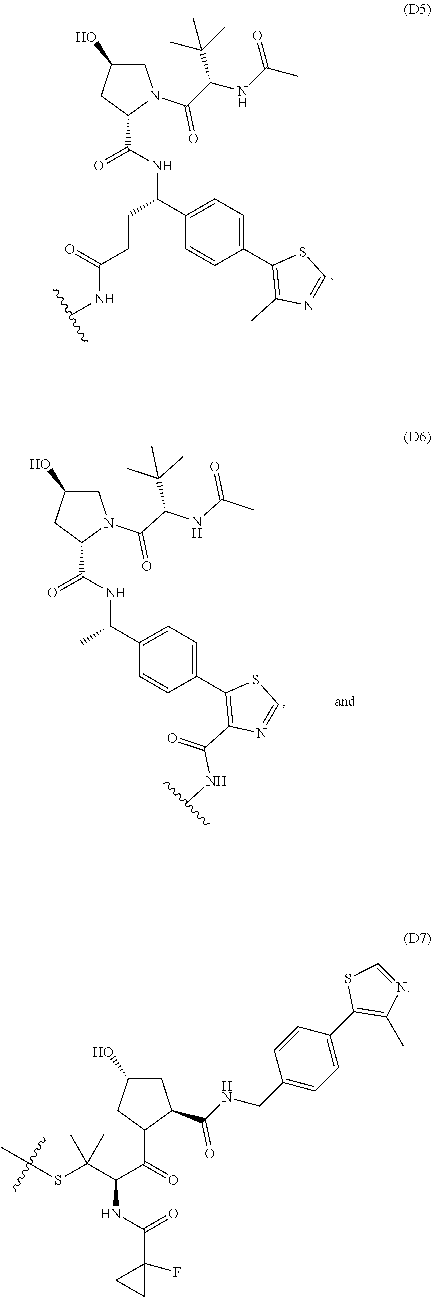 [SMILES: *NC(=O)CC[C@H](NC(=O)[C@@H]1C[C@@H](O)CN1C(=O)[C@@H](NC(C)=O)C(C)(C)C)c1ccc(-c2scnc2C)cc1 ; *NC(=O)c1ncsc1-c1ccc([C@H](C)NC(=O)[C@@H]2C[C@@H](O)CN2C(=O)[C@@H](NC(C)=O)C(C)(C)C)cc1 ; *SC(C)(C)[C@H](NC(=O)C1(F)CC1)C(=O)C1C[C@H](O)C[C@H]1C(=O)NCc1ccc(-c2scnc2C)cc1]

4. The bifunctional compound, or pharmaceutically acceptable salt or stereoisomer thereof of claim 1, wherein the degron binds an inhibitor of apoptosis protein (IAP).

5. The bifunctional compound, or pharmaceutically acceptable salt or stereoisomer thereof of claim 4, wherein the degron is represented by any one of the following structures:

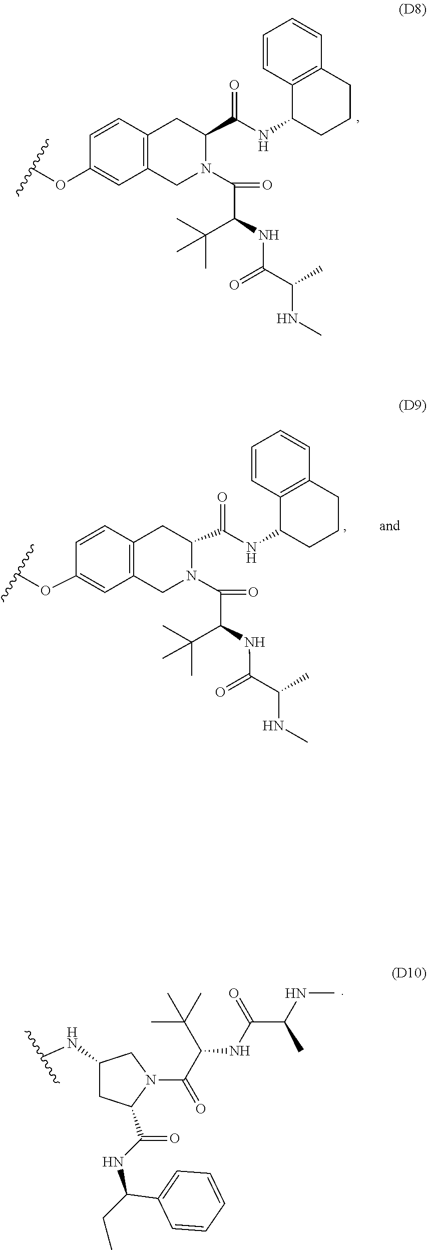

6. The bifunctional compound, or pharmaceutically acceptable salt or stereoisomer thereof of claim 1, wherein the degron binds cereblon (CRBN).

7. The bifunctional compound, or pharmaceutically acceptable salt or stereoisomer thereof of claim 6, wherein the degron is represented by any one of the following structures:

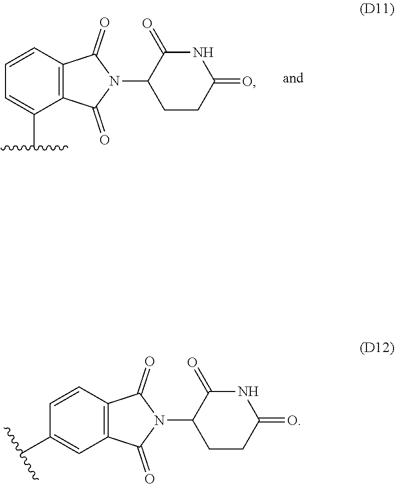 [SMILES: *c1cccc2c1C(=O)N(C1CCC(=O)NC1=O)C2=O ; *c1ccc2c(c1)C(=O)N(C1CCC(=O)NC1=O)C2=O]

8. The bifunctional compound, or pharmaceutically acceptable salt or stereoisomer thereof of claim 1, wherein the linker comprises an alkylene chain which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3-to 12-membered heterocyclene, 5-to 12-membered heteroarylene or any combination thereof; R′ is H or C₁-C₆ alkyl; and the interrupting and the one or both terminating groups may be the same or different, or
wherein the linker comprises a polyethylene glycol (PEG) chain which may terminate (at either or both termini) in at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3-to 12-membered heterocyclene, 5- to 12-membered heteroarylene or any combination thereof; R′ is H or C₁-C₆ alkyl; and the one or both terminating groups may be the same or different.

9. The bifunctional compound, or pharmaceutically acceptable salt or stereoisomer thereof of claim 8, wherein the alkylene chain comprises 1-6 alkylene units, or
wherein the polyethylene glycol chain comprises 1-6 PEG units.

10. (canceled)

11. (canceled)

12. The bifunctional compound, or pharmaceutically acceptable salt or stereoisomer thereof of claim 1, wherein the linker is represented by the structure: 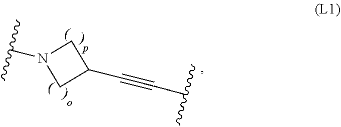
wherein o and p are independently 1, 2, or 3.

13. The bifunctional compound, or pharmaceutically acceptable salt or stereoisomer thereof of claim 1, wherein the linker comprises a C₁ to C₃ alkylene chain which may be interrupted by, and/or terminate (at either or both termini) in at least one of —O—, —N(R′)—, —C≡C—, —C(O)—, 4-to 8-membered heterocyclene, C₃-C₈ cycloalkyl, or any combination thereof: R′ is H or C₁-C₆ alkyl: and the interrupting and the one or both terminating groups may be the same or different.

14. The bifunctional compound, or pharmaceutically acceptable salt or stereoisomer thereof of claim 13, wherein the C₁ to C₃ alkylene chain terminates (at either or both termini) in at least one of —O—, —N(R′)—, —C≡C—, —C(O)—, or any combination thereof; and R′ is H or C₁-C₆ alkyl.

15. The bifunctional compound, or pharmaceutically acceptable salt or stereoisomer thereof of claim 14, wherein the linker is represented by any one of the structures: 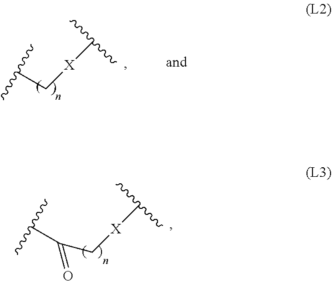
wherein n is 1, 2, or 3; and
X is —NH—, —O— or —C≡C—.

16. The bifunctional compound, or pharmaceutically acceptable salt or stereoisomer thereof of claim 13, wherein the C₁ to C₃ alkylene chain terminates (at either or both termini) in at least one of —C(O)—, 4-to 8-membered heterocyclene, or any combination thereof.

17. The bifunctional compound, or pharmaceutically acceptable salt or stereoisomer thereof of claim 16, wherein the linker is represented by the structure: 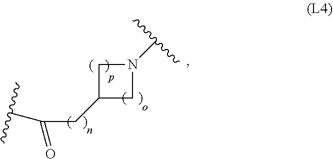
wherein n, o, and p are independently 1, 2, or 3.

18. The bifunctional compound, or pharmaceutically acceptable salt or stereoisomer thereof of claim 13, wherein the C₁ to C₃ alkylene chain terminates (at either or both termini) in at least one of —C≡C—, 4-to 8-membered heterocyclene, or any combination thereof.

19. The bifunctional compound, or pharmaceutically acceptable salt or stereoisomer thereof of claim 18, wherein the linker is represented by the structure: 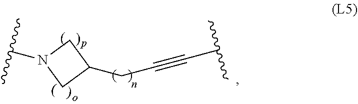
wherein n, o, and p are independently 1, 2, or 3.

20. The bifunctional compound, or pharmaceutically acceptable salt or stereoisomer thereof of claim 13, wherein the C₁ to C₃ alkylene chain terminates (at either or both termini) in at least one 4-to 8-membered heterocyclene.

21. The bifunctional compound, or pharmaceutically acceptable salt or stereoisomer thereof of claim 20, wherein the linker is represented by any one of the structures: 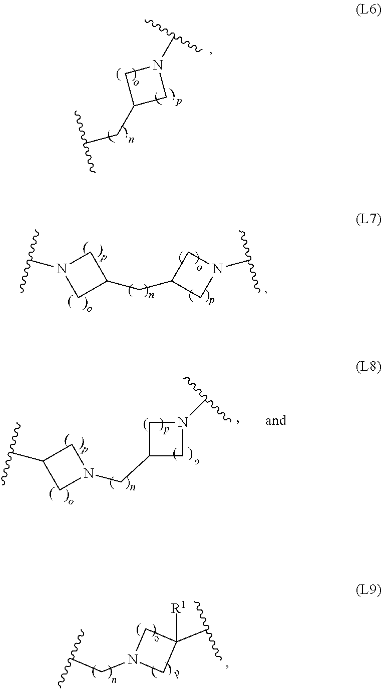
wherein each occurrence of n, o, and p is independently 1, 2, or 3; and
R¹ is —H, —OH, —NH₂, —SH, or —SeH.

22. The bifunctional compound, or pharmaceutically acceptable salt or stereoisomer thereof of claim 13, wherein the C₁ to C₃ alkylene chain is interrupted by a C₃-C₈ cycloalkyl.

23. The bifunctional compound, or pharmaceutically acceptable salt or stereoisomer thereof of claim 22, wherein the linker is represented by the structure: 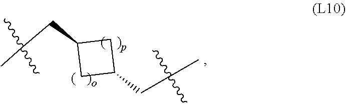
wherein o and p are independently 1, 2 or 3.

24. The bifunctional compound, or pharmaceutically acceptable salt or stereoisomer thereof of claim 1, which is represented by any one of structures: 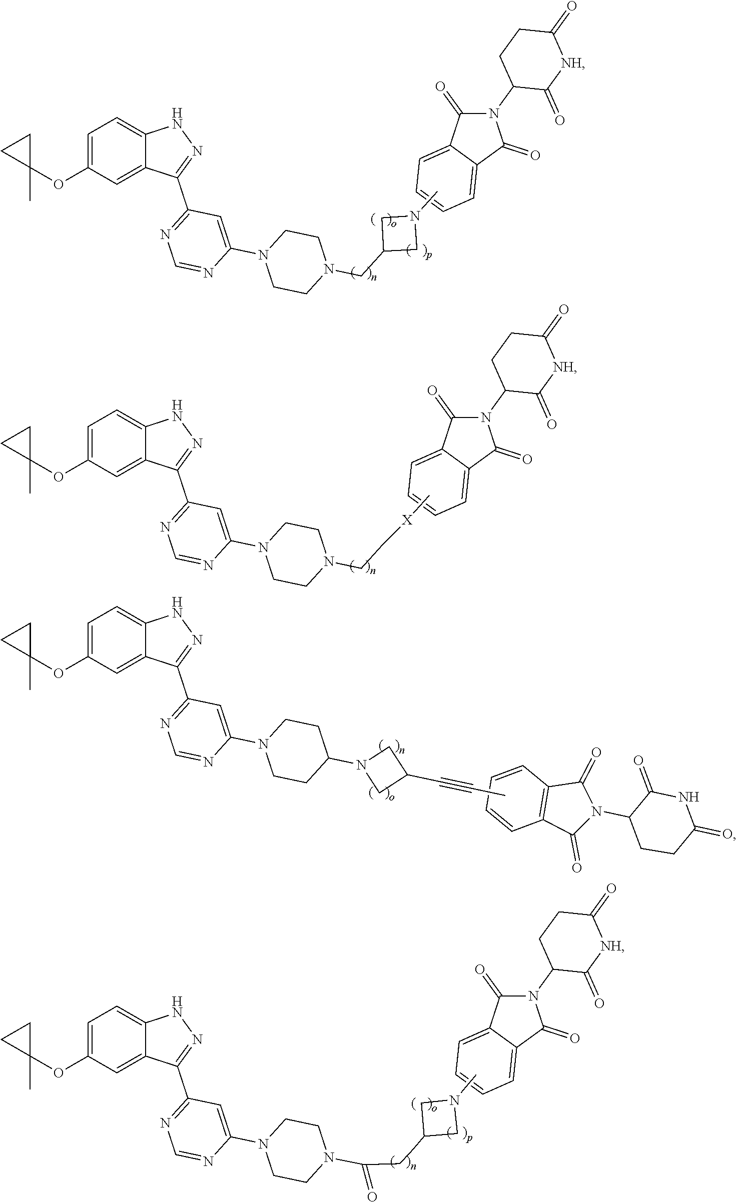 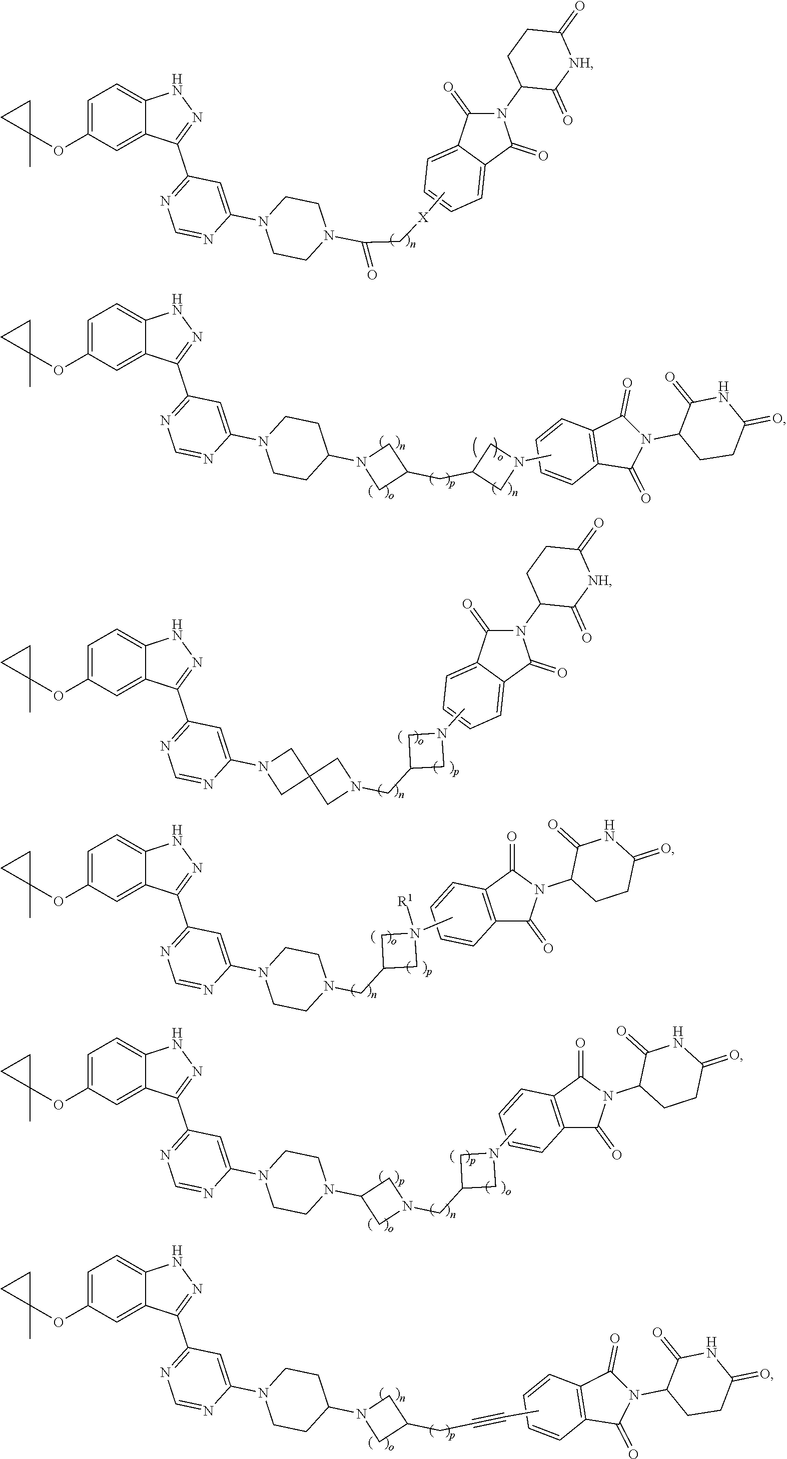 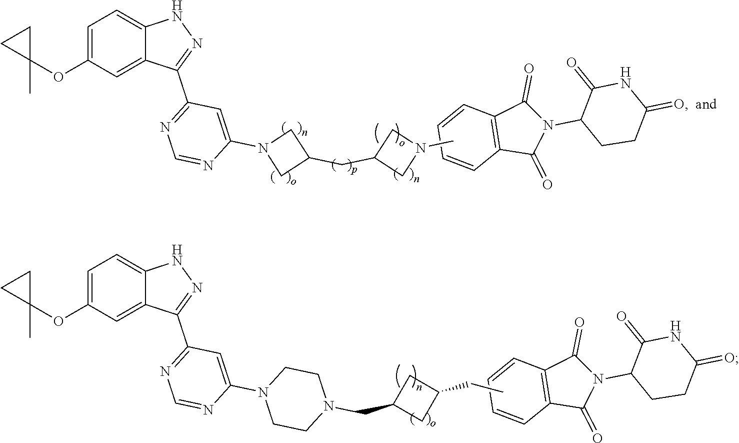
wherein n, o, and p are each independently 1, 2, or 3;
X is —NH—, —O— or alkynyl; and
R¹ is —H, —OH, —NH₂, —SH, or —ScH.

25. The bifunctional compound, or pharmaceutically acceptable salt or stereoisomer thereof of claim 1, which is represented by any one of the structures: 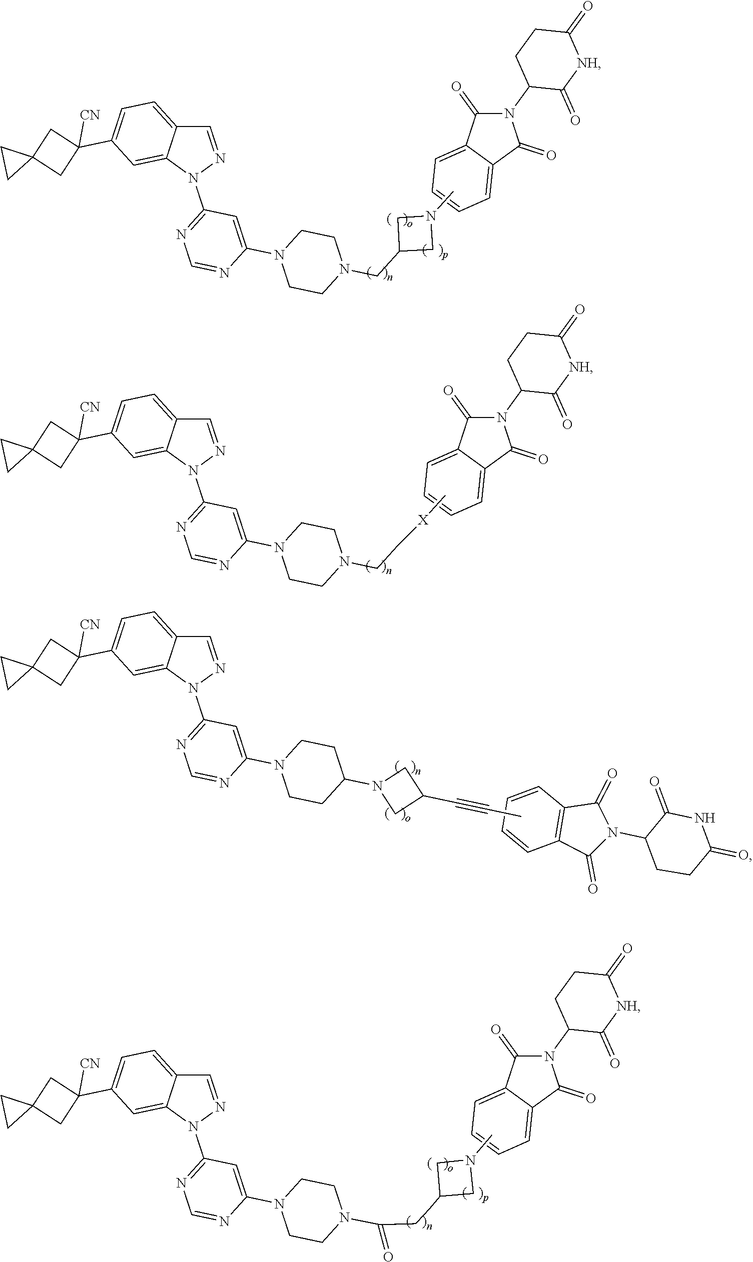 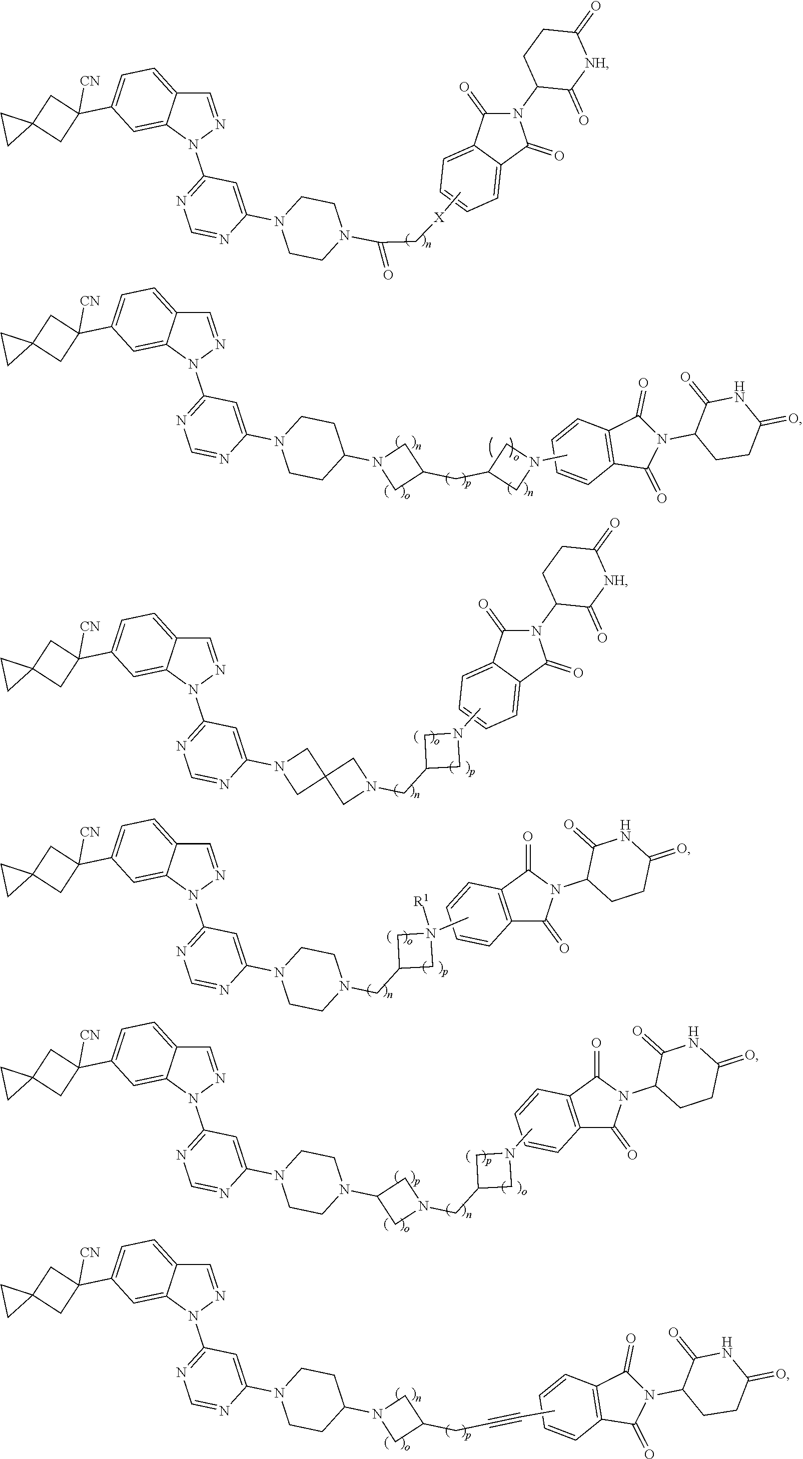 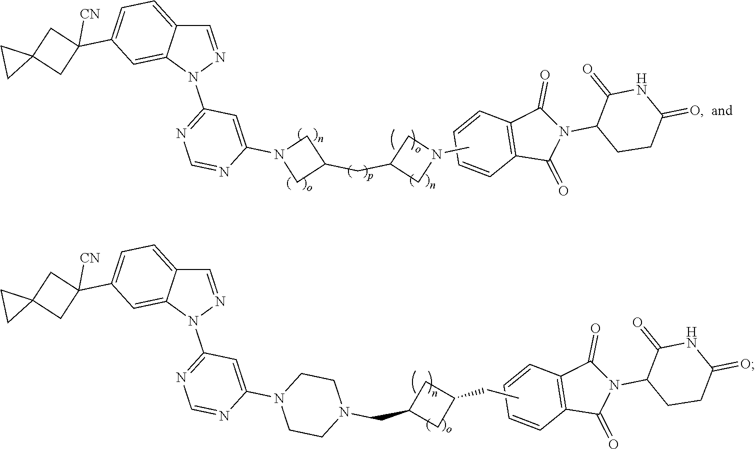
wherein n, o, and p are each independently 1, 2, or 3;
X is —NH—, —O— or alkynyl; and
R¹ is —H, —OH, —NH₂, —SH, or —SeH.

26. The bifunctional compound, or pharmaceutically acceptable salt or stereoisomer thereof of claim 1, wherein the linker is represented by any one of the structures:

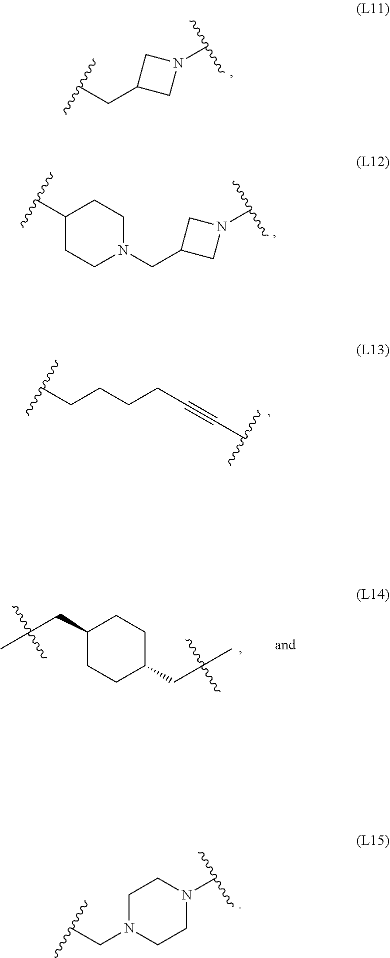 [SMILES: *CC1CN(*)C1 ; *C1CCN(CC2CN(*)C2)CC1 ; *C#CCCCC* ; *C[C@H]1CC[C@H](C*)CC1 ; *CN1CCN(*)CC1]

27. The bifunctional compound, or pharmaceutically acceptable salt or stereoisomer thereof of claim 1, which is:

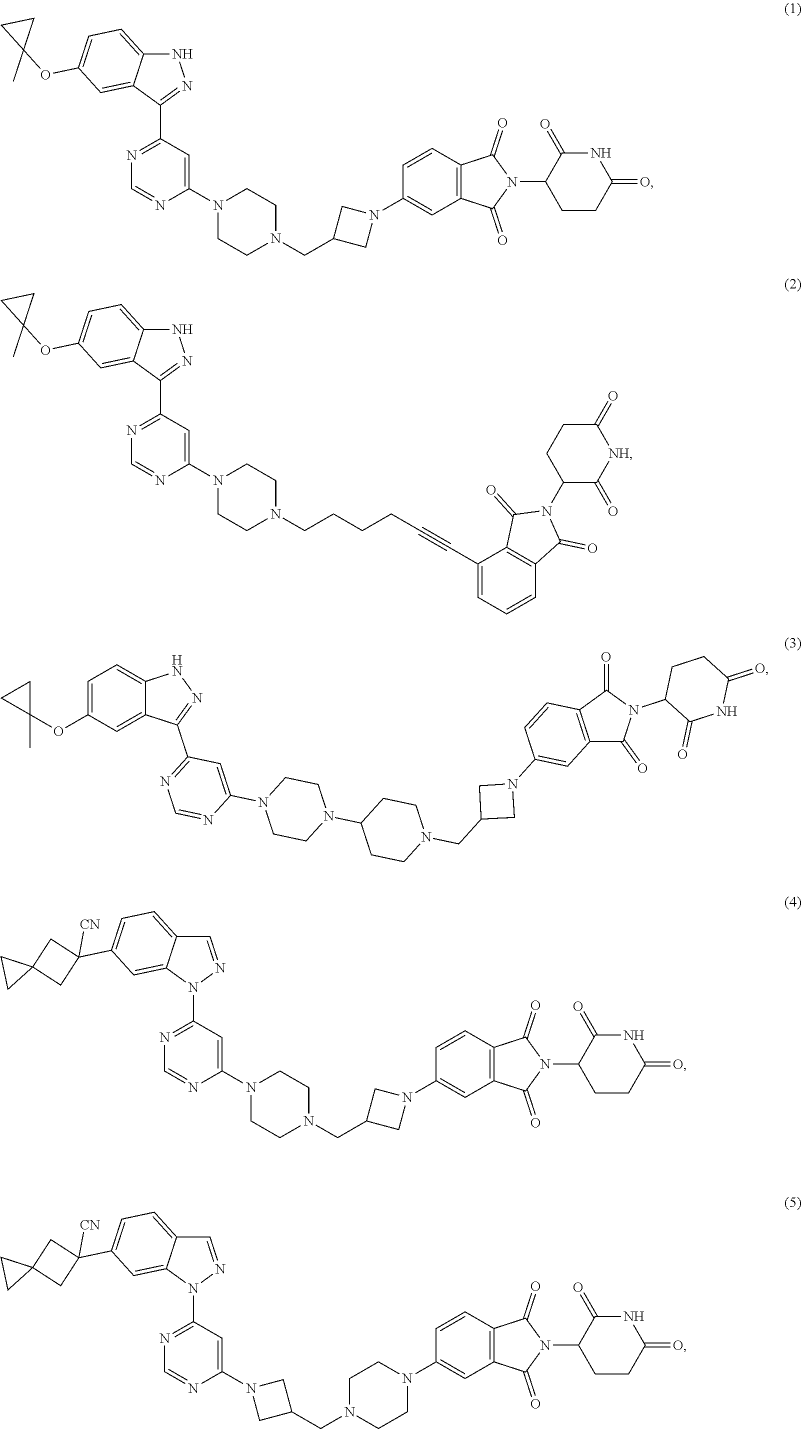 [SMILES: CC1(Oc2ccc3[nH]nc(-c4cc(N5CCN(CC6CN(c7ccc8c(c7)C(=O)N(C7CCC(=O)NC7=O)C8=O)C6)CC5)ncn4)c3c2)CC1 ; CC1(Oc2ccc3[nH]nc(-c4cc(N5CCN(CCCCC#Cc6cccc7c6C(=O)N(C6CCC(=O)NC6=O)C7=O)CC5)ncn4)c3c2)CC1 ; CC1(Oc2ccc3[nH]nc(-c4cc(N5CCN(C6CCN(CC7CN(c8ccc9c(c8)C(=O)N(C8CCC(=O)NC8=O)C9=O)C7)CC6)CC5)ncn4)c3c2)CC1 ; N#CC1(c2ccc3cnn(-c4cc(N5CCN(CC6CN(c7ccc8c(c7)C(=O)N(C7CCC(=O)NC7=O)C8=O)C6)CC5)ncn4)c3c2)CC2(CC2)C1 ; N#CC1(c2ccc3cnn(-c4cc(N5CC(CN6CCN(c7ccc8c(c7)C(=O)N(C7CCC(=O)NC7=O)C8=O)CC6)C5)ncn4)c3c2)CC2(CC2)C1]

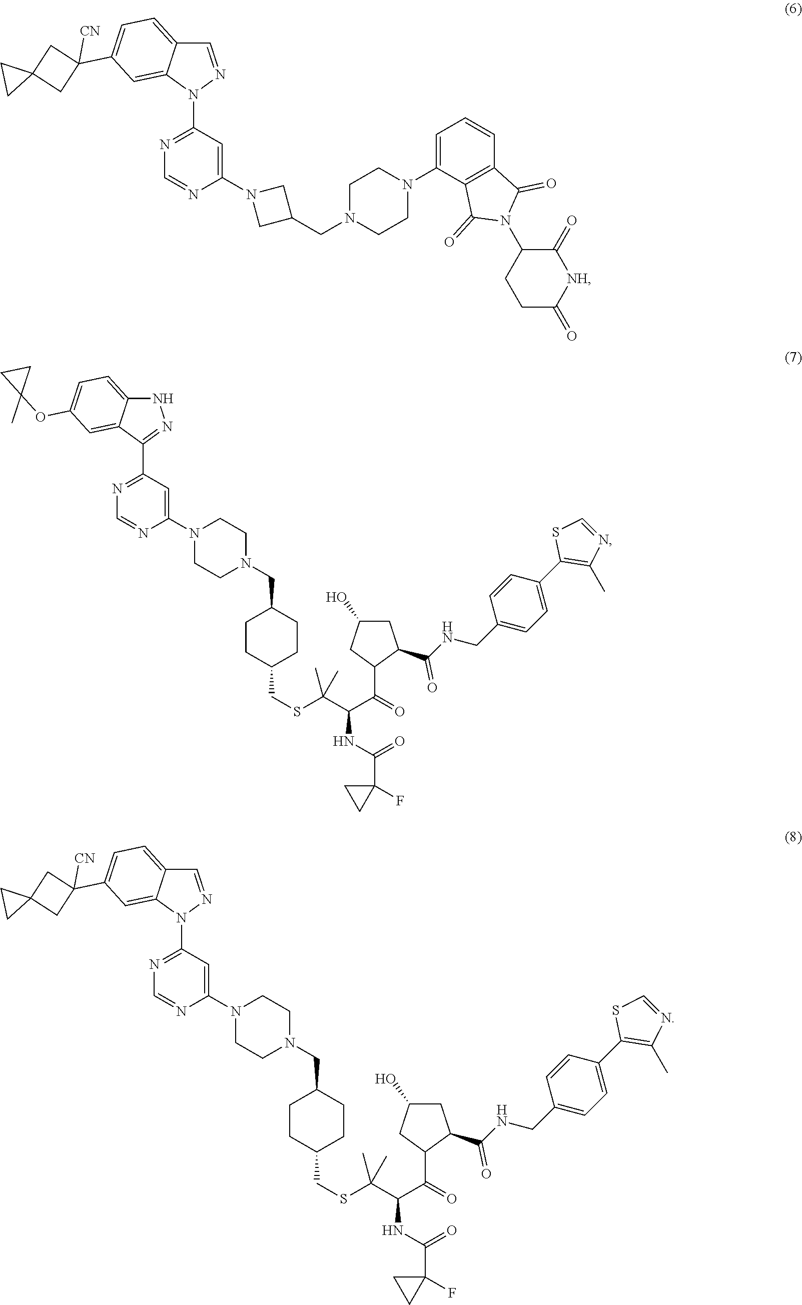 [SMILES: N#CC1(c2ccc3cnn(-c4cc(N5CC(CN6CCN(c7cccc8c7C(=O)N(C7CCC(=O)NC7=O)C8=O)CC6)C5)ncn4)c3c2)CC2(CC2)C1 ; Cc1ncsc1-c1ccc(CNC(=O)[C@@H]2C[C@@H](O)CC2C(=O)[C@@H](NC(=O)C2(F)CC2)C(C)(C)SC[C@H]2CC[C@H](CN3CCN(c4cc(-c5n[nH]c6ccc(OC7(C)CC7)cc56)ncn4)CC3)CC2)cc1 ; Cc1ncsc1-c1ccc(CNC(=O)[C@@H]2C[C@@H](O)CC2C(=O)[C@@H](NC(=O)C2(F)CC2)C(C)(C)SC[C@H]2CC[C@H](CN3CCN(c4cc(-n5ncc6ccc(C7(C#N)CC8(CC8)C7)cc65)ncn4)CC3)CC2)cc1]

28. A pharmaceutical composition, comprising a therapeutically effective amount of the bifunctional compound or pharmaceutically acceptable salt or stereoisomer thereof of claim 1, and a pharmaceutically acceptable carrier.

29.-31. (canceled)

32. A method of treating a disease or disorder that is characterized by aberrant activity of LRRK2, comprising administering to a subject in need thereof a therapeutically effective amount of the bifunctional compound or pharmaceutically acceptable salt or stereoisomer thereof of claim 1.

33. The method of claim 32, wherein the disease or disorder is a neurodegenerative disease or disorder, an inflammatory bowel disease, Crohn's disease, leprosy (Hansen's disease), tuberculosis, meningioma, or a cancer.

34. The method of claim 33, wherein the neurodegenerative disease or disorder is Parkinson's disease, or
wherein the cancer is breast cancer, lung cancer or thyroid cancer.

35.-34. (canceled)

42. A method of reducing the levels of LRRK2 in a cell, either in vitro or in vivo, comprising contacting the cell with an effective amount of the bifunctional compound or pharmaceutically acceptable salt or stereoisomer thereof of claim 1.


SEQUENCE LISTING (1 sequence)

SEQ ID NO: 1 - protein, 2527 aa, Homo sapiens
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE